(19) United States
(12) Patent Application Publication
(10) Pub. No.: US 2025/0332075 A1
(43) Pub. Date: Oct. 30, 2025

(54) POLYAMIDE MICROCAPSULES

(71) Applicant: Firmenich SA, Satigny (CH)

(72) Inventors: Ang LI, Plainsboro, NJ (US); Christopher HANSEN, Plainsboro, NJ (US); Huda JERRI, Plainsboro, NJ (US)

(21) Appl. No.: 18/863,440

(22) PCT Filed: May 2, 2023
(86) PCT No.: PCT/EP2023/061559

§ 371 Date: Nov. 6, 2024

Related U.S. Application Data
(60) Provisional application No. 63/364,388, filed on May 9, 2022.

(30) Foreign Application Priority Data
May 18, 2022 (EP) 22173941.0

(51) Int. Cl. A61K 8/11; A61K 8/88; A61Q 5/02; A61Q 5/06; A61Q 5/12; A61Q 11/00; A61Q 15/00; A61Q 19/00; A61Q 19/10; C11D 3/12; C11D 3/34; C11D 3/37; C11D 3/386; C11D 3/50; C11D 17/00

(52) U.S. Cl. CPC A61K 8/11; A61K 8/88; A61Q 5/02; A61Q 5/065; A61Q 5/12; A61Q 11/00; A61Q 15/00; A61Q 19/007; A61Q 19/10; C11D 3/1246; C11D 3/3409; C11D 3/3719; C11D 3/386; C11D 3/505; C11D 17/0039

(57) ABSTRACT

Disclosed herein is a new process for the preparation of polyamide microcapsules. Also disclosed herein are the polyamide microcapsules. Also disclosed herein are Perfuming compositions and consumer products including the microcapsules, including in the form of home care or personal care products.

### TECHNICAL FIELD

The present invention relates to a new process for the preparation of polyamide microcapsules. Polyamide microcapsules are also an object of the invention. Perfuming compositions and consumer products comprising said microcapsules, in particular perfumed consumer products in the form of home care or personal care products, are also part of the invention.

### BACKGROUND OF THE INVENTION

One of the problems faced by the perfumery industry lies in the relatively rapid loss of olfactive benefit provided by odoriferous compounds due to their volatility, particularly that of “top-notes”. In order to tailor the release rates of volatiles, delivery systems such as microcapsules containing a perfume are needed to protect and later release the core payload when triggered. A key requirement from the industry regarding these systems is to survive suspension in challenging bases without physically dissociating or degrading. This is referred to as stability for the delivery system. For instance, fragranced personal and household cleansers containing high levels of aggressive surfactant detergents are very challenging for the stability of microcapsules.

In addition to the performance in terms of stability and olfactive performance, the consumer demand for eco-friendly delivery systems is more and more important and is driving the development of new delivery systems.

There is therefore still a need to provide new microcapsules using more eco-friendly materials, while not compromising on the performance of the microcapsules, in particular in terms of stability in a challenging medium such as a consumer product base, as well as in delivering a good performance in terms of active ingredient delivery, e.g. olfactive performance in the case of perfuming ingredients.

The present invention is proposing a solution to the above-mentioned problem by providing new polyamide microcapsules and a process for preparing said microcapsules.

### SUMMARY OF THE INVENTION

It has now been surprisingly found that performing core-shell microcapsules encapsulating hydrophobic material could be obtained by reacting at least one acyl chloride with at least one co-oligopeptide. The process of the invention therefore provides a solution to the above-mentioned problems as it allows preparing microcapsules with the desired stability in challenging bases.

A first object of the invention is a polyamide core-shell microcapsule comprising:
a core comprising a hydrophobic material, preferably a perfume oil, and
a polyamide shell comprising the reaction product between at least one acyl chloride and at least one co-oligopeptide.

Another object of the invention is a slurry comprising the microcapsules as defined above.

Another object of the invention is a process for preparing a polyamide core-shell microcapsule slurry comprising the steps of:
a) Dispersing an oil phase comprising a hydrophobic material and at least one acyl chloride into a dispersing phase to form a two-phases dispersion;
b) Performing a curing step to form microcapsules in the form of a slurry;
   wherein at least one stabilizer is added in the oil phase and/or in the dispersing phase, and
   wherein at least one co-oligopeptide is added in the dispersing phase and/or in the oil phase and/or in the two-phases dispersion.

Another object of the invention is a polyamide core-shell microcapsule slurry obtainable by the process as defined above.

A perfuming composition comprising:
(i) microcapsule or a microcapsule slurry as defined above, wherein the hydrophobic material comprises a perfume,
(ii) at least one ingredient selected from the group consisting of a perfumery carrier and a perfumery base
(iii) optionally at least one perfumery adjuvant
   is another object of the invention.

Another object of the invention is a consumer product comprising:
a personal care active base, and
microcapsules or a microcapsule slurry as defined above or the perfuming composition as defined above,
   wherein the consumer product is in the form of a personal care composition.

Another object of the invention is a consumer product comprising:
a home care or a fabric care active base, and
microcapsules or a microcapsule slurry as defined above or the perfuming composition as defined above,
wherein the consumer product is in the form of a home care or a fabric care composition.

### DETAILED DESCRIPTION OF THE INVENTION

Unless stated otherwise, percentages (%) are meant to designate a percentage by weight of a composition.

By “active ingredient”, it is meant a single compound or a combination of ingredients.

By “perfume or flavour oil”, it is meant a single perfuming or flavouring compound or a mixture of several perfuming or flavouring compounds.

By “consumer product” or “end-product” it is meant a manufactured product ready to be distributed, sold and used by a consumer.

For the sake of clarity, by the expression “dispersion” in the present invention it is meant a system in which particles are dispersed in a continuous phase of a different composition and it specifically includes a suspension or an emulsion.

A “microcapsule”, or the similar, in the present invention it is meant that core-shell microcapsules have a particle size distribution in the micron range (e.g. a mean diameter (d(v, 0.5)) comprised between about 1 and 3000 microns, preferably between 1 and 500 microns) and comprise an external solid polyamide-based shell and an internal continuous oil phase enclosed by the external shell.

By “microcapsule slurry”, it is meant microcapsule(s) that is (are) dispersed in a liquid. According to an embodiment, the slurry is an aqueous slurry, i.e. the microcapsule(s) is (are) dispersed in an aqueous phase.

By “polyamide microcapsules”, it means that the microcapsule's shell comprises a polyamide material. The wording “polyamide microcapsules” can also encompass a shell made of a composite comprising a polyamide material and another material, for example a polymer (like a protein).

“Polyamide-based microcapsules” and “polyamide microcapsules” are used indifferently in the present invention.

By “salt”, it means an ionic compound that can dissolve in the dispersing phase (typically water) and form metal ions with one or more than one valency.

The wording “dispersing phase” and “continuous phase” can be used indifferently in the present invention.

It has been found that polyamide core-shell microcapsules with overall good performance in challenging bases could be obtained when the shell comprises the reaction product between at least one acyl chloride and at least one co-oligopeptide.

### FIGURES

FIG. 1 represents a SEM image of microcapsules (A) according to the invention.

FIG. 2 represents a SEM image of microcapsules (D) according to the invention.

### POLYAMIDE CAPSULE

A first object of the invention is a polyamide core-shell microcapsule comprising:
a core comprising a hydrophobic material, preferably a perfume oil, and
a polyamide shell comprising the reaction product between at least one acyl chloride and at least one co-oligopeptide.

Another object of the invention is a polyamide core-shell microcapsule slurry comprising at least one polyamide core-shell microcapsule, wherein the microcapsule comprises:
a core comprising a hydrophobic material, preferably a perfume oil, and
a polyamide shell comprising the reaction product between at least one acyl chloride and at least one co-oligopeptide.

According to an embodiment, the polyamide shell comprising the reaction product between:
at least one acyl chloride,
at least one co-oligopeptide,
optionally at least one oligopeptide, and
optionally at least one free amino acid.

According to an embodiment, the weight ratio between free amino acid, if present, and co-oligopeptide is comprised between 0.01:1 to 40:1.

According to an embodiment, the weight ratio between oligopeptide, if present, and co-oligopeptide is comprised between 0.01:1 to 40:1.

### Hydrophobic Material

The hydrophobic material according to the invention can be “inert” material like solvents or active ingredients. The core is preferably an oil-based core.

By “hydrophobic material”, it is meant any hydrophobic material which forms a two-phase dispersion when mixed with the dispersing phase (typically water). The hydrophobic material is typically liquid at about 20° C.

According to an embodiment, the hydrophobic material is a hydrophobic active ingredient.

When hydrophobic materials are active ingredients, they are preferably chosen from the group consisting of flavors, flavor ingredients, perfumes, perfume ingredients, nutraceuticals, cosmetics, pest control agents, biocide actives and mixtures thereof.

According to a particular embodiment, the hydrophobic material comprises a mixture of a perfume with another ingredient selected from the group consisting of nutraceuticals, cosmetics, pest control agents and biocide actives.

According to a particular embodiment, the hydrophobic material comprises a mixture of biocide actives with another ingredient selected from the group consisting of perfumes, nutraceuticals, cosmetics, pest control agents.

According to a particular embodiment, the hydrophobic material comprises a mixture of pest control agents with another ingredient selected from the group consisting of perfumes, nutraceuticals, cosmetics, biocide actives.

According to a particular embodiment, the hydrophobic material comprises a perfume.

According to a particular embodiment, the hydrophobic material consists of a perfume.

According to a particular embodiment, the hydrophobic material consists of biocide actives.

According to a particular embodiment, the hydrophobic material consists of pest control agents.

By “perfume” (or also “perfume oil”) what is meant here is an ingredient or a composition that is a liquid at about 20° C. According to any one of the above embodiments said perfume oil can be a perfuming ingredient alone or a mixture of ingredients in the form of a perfuming composition. As a “perfuming ingredient” it is meant here a compound, which is used for the primary purpose of conferring or modulating an odor. In other words such an ingredient, to be considered as being a perfuming one, must be recognized by a person skilled in the art as being able to at least impart or modify in a positive or pleasant way the odor of a composition, and not just as having an odor. For the purpose of the present invention, perfume oil also includes a combination of perfuming ingredients with substances which together improve, enhance or modify the delivery of the perfuming ingredients, such as perfume precursors, emulsions or dispersions, as well as combinations which impart an additional benefit beyond that of modifying or imparting an odor, such as long-lastingness, blooming, malodor counteraction, antimicrobial effect, microbial stability, pest control.

The nature and type of the perfuming ingredients present in the oil phase do not warrant a more detailed description here, which in any case would not be exhaustive, the skilled person being able to select them on the basis of its general knowledge and according to intended use or application and the desired organoleptic effect. In general terms, these perfuming ingredients belong to chemical classes as varied as alcohols, aldehydes, ketones, esters, ethers, acetates, nitriles, terpenoids, nitrogenous or sulfurous heterocyclic compounds and essential oils, and said perfuming co-ingredients can be of natural or synthetic origin. Many of these co-ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery.

In particular one may cite perfuming ingredients which are commonly used in perfume formulations, such as:
Aldehydic ingredients: decanal, dodecanal, 2-methyl-undecanal, 10-undecenal, octanal, nonanal and/or nonenal;
Aromatic-herbal ingredients: eucalyptus oil, camphor, eucalyptol, 5-methyltricyclo[6.2.1.0˜2,7˜]undecan-4-one, 1-methoxy-3-hexanethiol, 2-ethyl-4,4-dimethyl-1,3-oxathiane, 2,2,7/8,9/10-Tetramethylspiro[5.5]undec-8-en-1-one, menthol and/or alpha-pinene;
Balsamic ingredients: coumarin, ethylvanillin and/or vanillin;
Citrus ingredients: dihydromyrcenol, citral, orange oil, linalyl acetate, citronellyl nitrile, orange terpenes, limonene, 1-p-menthen-8-yl acetate and/or 1,4(8)-p-menthadiene;
Floral ingredients: methyl dihydrojasmonate, linalool, citronellol, phenylethanol, 3-(4-tert-butylphenyl)-2-methylpropanal, hexylcinnamic aldehyde, benzyl acetate, benzyl salicylate, tetrahydro-2-isobutyl-4-methyl-4(2H)-pyranol, beta ionone, methyl 2-(methylamino)benzoate, (E)-3-methyl-4-(2,6,6-trimethyl-2-cyclohexen-1-yl)-3-buten-2-one, (1E)-1-(2,6,6-trimethyl-2-cyclohexen-1-yl)-1-penten-3-one, 1-(2,6,6-trimethyl-1,3-cyclohexadien-1-yl)-2-buten-1-one, (2E)-1-(2,6,6-trimethyl-2-cyclohexen-1-yl)-2-buten-1-one, (2E)-1-[2,6,6-trimethyl-3-cyclohexen-1-yl]-2-buten-1-one, (2E)-1-(2,6,6-trimethyl-1-cyclohexen-1-yl)-2-buten-1-one, 2,5-dimethyl-2-indanmethanol, 2,6,6-trimethyl-3-cyclohexene-1-carboxylate, 3-(4,4-dimethyl-1-cyclohexen-1-yl)propanal, hexyl salicylate, 3,7-dimethyl-1,6-nonadien-3-ol, 3-(4-isopropylphenyl)-2-methylpropanal, verdyl acetate, geraniol, p-menth-1-en-8-ol, 4-(1,1-dimethylethyl)-1-cyclohexyle acetate, 1,1-dimethyl-2-phenylethyl acetate, 4-cyclohexyl-2-methyl-2-butanol, amyl salicylate, high cis methyl dihydrojasmonate, 3-methyl-5-phenyl-1-pentanol, verdyl proprionate, geranyl acetate, tetrahydro linalool, cis-7-p-menthanol, propyl (S)-2-(1,1-dimethylpropoxy)propanoate, 2-methoxynaphthalene, 2,2,2-trichloro-1-phenylethyl acetate, 4/3-(4-hydroxy-4-methylpentyl)-3-cyclohexene-1-carbaldehyde, amylcinnamic aldehyde, 8-decen-5-olide, 4-phenyl-2-butanone, isononyle acetate, 4-(1,1-dimethylethyl)-1-cyclohexyl acetate, verdyl isobutyrate and/or mixture of methylionones isomers;
Fruity ingredients: gamma-undecalactone, 2,2,5-trimethyl-5-pentylcyclopentanone, 2-methyl-4-propyl-1,3-oxathiane, 4-decanolide, ethyl 2-methyl-pentanoate, hexyl acetate, ethyl 2-methylbutanoate, gamma-nonalactone, allyl heptanoate, 2-phenoxyethyl isobutyrate, ethyl 2-methyl-1,3-dioxolane-2-acetate, 3-(3,3/1,1-dimethyl-5-indanyl)propanal, diethyl 1,4-cyclohexanedicarboxylate, 3-methyl-2-hexen-1-yl acetate, 1-[3,3-dimethylcyclohexyl]ethyl [3-ethyl-2-oxiranyl]acetate and/or diethyl 1,4-cyclohexane dicarboxylate;
Green ingredients: 2-methyl-3-hexanone (E)-oxime, 2,4-dimethyl-3-cyclohexene-1-carbaldehyde, 2-tert-butyl-1-cyclohexyl acetate, styrallyl acetate, allyl (2-methylbutoxy)acetate, 4-methyl-3-decen-5-ol, diphenyl ether, (Z)-3-hexen-1-ol and/or 1-(5,5-dimethyl-1-cyclohexen-1-yl)-4-penten-1-one;
Musk ingredients: 1,4-dioxa-5,17-cycloheptadecanedione, (Z)-4-cyclopentadecen-1-one, 3-methylcyclopentadecanone, 1-oxa-12-cyclohexadecen-2-one, 1-oxa-13-cyclohexadecen-2-one, (9Z)-9-cycloheptadecen-1-one, 2-{1S)-1-[(1R)-3,3-dimethylcyclohexyl]ethoxy}-2-oxoethyl propionate 3-methyl-5-cyclopentadecen-1-one, 1,3,4,6,7,8-hexahydro-4,6,6,7,8,8-hexamethyl-cyclopenta-g-2-benzopyrane, (1S,1′R)-2-[1-(3′,3′-dimethyl-1′-cyclohexyl)ethoxy]-2-methylpropyl propanoate, oxacyclohexadecan-2-one and/or (1S,1′R)-[1-(3′,3′-dimethyl-1′-cyclohexyl)ethoxycarbonyl]methyl propanoate;
Woody ingredients: 1-[(1RS,6SR)-2,2,6-trimethylcyclohexyl]-3-hexanol, 3,3-dimethyl-5-[(1R)-2,2,3-trimethyl-3-cyclopenten-1-yl]-4-penten-2-ol, 3,4′-dimethylspiro[oxirane-2,9′-tricyclo[6.2.1.02,7]undec[4]ene, (1-ethoxyethoxy)cyclododecane, 2,2,9,11-tetramethylspiro[5.5]undec-8-en-1-yl acetate, 1-(octahydro-2,3,8,8-tetramethyl-2-naphtalenyl)-1-ethanone, patchouli oil, terpenes fractions of patchouli oil, Clearwood®, (1′R,E)-2-ethyl-4-(2′,2′,3′-trimethyl-3′-cyclopenten-1′-yl)-2-buten-1-ol, 2-ethyl-4-(2,2,3-trimethyl-3-cyclopenten-1-yl)-2-buten-1-ol, methyl cedryl ketone, 5-(2,2,3-trimethyl-3-cyclopentenyl)-3-methylpentan-2-ol, 1-(2,3,8,8-tetramethyl-1,2,3,4,6,7,8,8a-octahydronaphthalen-2-yl)ethan-1-one and/or isobornyl acetate;

Other ingredients (e.g. amber, powdery spicy or watery): dodecahydro-3a,6,6,9a-tetramethyl-naphtho[2,1-b]furan and any of its stereoisomers, heliotropin, anisic aldehyde, eugenol, cinnamic aldehyde, clove oil, 3-(1,3-benzodioxol-5-yl)-2-methylpropanal, 7-methyl-2H-1,5-benzodioxepin-3(4H)-one, 2,5,5-trimethyl-1,2,3,4,4a,5,6,7-octahydro-2-naphthalenol, 1-phenylvinyl acetate, 6-methyl-7-oxa-1-thia-4-azaspiro[4.4]nonan and/or 3-(3-isopropyl-1-phenyl)butanal.

According to a particular embodiment, the perfume or perfume formulation comprises a fragrance modulator (that can be used in addition to the hydrophobic solvent when present or as substitution of the hydrophobic solvent when there is no hydrophobic solvent).

Preferably, the fragrance modulator is defined as a fragrance material with
a vapor pressure of less than 0.0008 Torr at 22° C.;
a c log P of 3.5 and higher, preferably 4.0 and higher and more preferably 4.5
at least two Hansen solubility parameters selected from a first group consisting of: an atomic dispersion force from 12 to 20, a dipole moment from 1 to 7, and a hydrogen bonding from 2.5 to 11,
at least two Hansen solubility parameters selected from a second group consisting of: an atomic dispersion force from 14 to 20, a dipole moment from 1 to 8, and a hydrogen bonding from 4 to 11, when in solution with a compound having a vapor pressure range of 0.0008 to 0.08 Torr at 22° C.

Preferably as examples the following ingredients can be listed as fragrance modulators but the list in not limited to the following materials: alcohol C12, oxacyclohexadec-12/13-en-2-one, 3-[(2′,2′,3′-trimethyl-3′-cyclopenten-1′-yl)methoxy]-2-butanol, cyclohexadecanone, (Z)-4-cyclopentadecen-1-one, cyclopentadecanone, (8Z)-oxacycloheptadec-8-en-2-one, 2-[5-(tetrahydro-5-methyl-5-vinyl-2-furyl)-tetrahydro-5-methyl-2-furyl]-2-propanol, muguet aldehyde, 1,5,8-trimethyl-13-oxabicyclo[10.1.0]trideca-4,8-diene, (+−)-4,6,6,7,8,8-hexamethyl-1,3,4,6,7,8-hexahydrocyclopenta[g]isochromene, (+)-(1S,2S,3S,5R)-2,6,6-trimethylspiro[bicyclo[3.1.1]heptane-3,1′-cyclohexane]-2′-en-4′-one, oxacyclohexadecan-2-one, 2-{(1S)-1-[(1R)-3,3-dimethylcyclohexyl]ethoxy}-2-oxoethyl propionate, (+)-(4R,4aS,6R)-4,4a-dimethyl-6-(1-propen-2-yl)-4,4a,5,6,7,8-hexahydro-2(3H)-naphthalenone, amylcinnamic aldehyde, hexylcinnamic aldehyde, hexyl salicylate, (1E)-1-(2,6,6-trimethyl-1-cyclohexen-1-yl)-1,6-heptadien-3-one, (9Z)-9-cycloheptadecen-1-one.

It is also understood that said ingredients may also be compounds known to release in a controlled manner various types of perfuming compounds also known as properfume or profragrance. Non-limiting examples of suitable properfumes may include 4-(dodecylthio)-4-(2,6,6-trimethyl-2-cyclohexen-1-yl)-2-butanone, 4-(dodecylthio)-4-(2,6,6-trimethyl-1-cyclohexen-1-yl)-2-butanone, 3-(dodecylthio)-1-(2,6,6-trimethyl-3-cyclohexen-1-yl)-1-butanone, 2-(dodecylthio)octan-4-one, 2-phenylethyl oxo(phenyl)acetate, 3,7-dimethylocta-2,6-dien-1-yl oxo(phenyl)acetate, (Z)-hex-3-en-1-yl oxo(phenyl)acetate, 3,7-dimethyl-2,6-octadien-1-yl hexadecanoate, bis(3,7-dimethylocta-2,6-dien-1-yl) succinate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methylundec-1-ene, (2-((2-methylundec-1-en-1-yl)oxy)ethoxy)benzene, 2-methyl-1-(octan-3-yloxy)undec-1-ene, 1-methoxy-4-(1-phenethoxyprop-1-en-2-yl)benzene, 1-methyl-4-(1-phenethoxyprop-1-en-2-yl)benzene, 2-(1-phenethoxyprop-1-en-2-yl)naphthalene, (2-phenethoxyvinyl)benzene, 2-(1-((3,7-dimethyloct-6-en-1-yl)oxy)prop-1-en-2-yl)naphthalene, (2-((2-pentylcyclopentylidene)methoxy)ethyl)benzene, 4-allyl-2-methoxy-1-((2-methoxy-2-phenylvinyl)oxy)benzene, (2-((2-heptylcyclopentylidene)methoxy)ethyl)benzene, 1-isopropyl-4-methyl-2-((2-pentylcyclopentylidene)methoxy)benzene, 2-methoxy-1-((2-pentylcyclopentylidene)methoxy)-4-propylbenzene, 3-methoxy-4-((2-methoxy-2-phenylvinyl)oxy)benzaldehyde, 4-((2-(hexyloxy)-2-phenylvinyl)oxy)-3-methoxybenzaldehyde or a mixture thereof.

The perfuming ingredients may be dissolved in a solvent of current use in the perfume industry. The solvent is preferably not an alcohol. Examples of such solvents are diethyl phthalate, isopropyl myristate, Abalyn® (rosin resins, available from Eastman), benzyl benzoate, ethyl citrate, limonene or other terpenes, or isoparaffins. Preferably, the solvent is very hydrophobic and highly sterically hindered, like for example Abalyn® or benzyl benzoate. Preferably the perfume comprises less than 30% of solvent. More preferably the perfume comprises less than 20% and even more preferably less than 10% of solvent, all these percentages being defined by weight relative to the total weight of the perfume. Most preferably, the perfume is essentially free of solvent.

According to a particular embodiment, the perfume comprises at least 35% of perfuming ingredients having a log P above 3.

Log P is the common logarithm of estimated octanol-water partition coefficient, which is known as a measure of lipophilicity.

The Log P values of many perfuming compound have been reported, for example, in the Pomona92 database, available from Daylight Chemical Information Systems, Inc. (Daylight CIS), Irvine, Calif., which also contains citations to the original literature. Log P values are most conveniently calculated by the “C LOG P” program, also available from Daylight CIS. This program also lists experimental log P values when they are available in the Pomona92 database. The “calculated log P” (c Log P) is determined by the fragment approach of Hansch and Leo (cf., A. Leo, in Comprehensive Medicinal Chemistry, Vol. 4, C. Hansch, P. G. Sammens, J. B. Taylor and C. A. Ramsden, Eds., p. 295, Pergamon Press, 1990). The fragment approach is based on the chemical structure of each perfume oil ingredient, and takes into account the numbers and types of atoms, the atom connectivity, and chemical bonding. The c Log P values, which are the most reliable and widely used estimates for this physicochemical property, are preferably used instead of the experimental Log P values in the selection of perfuming compounds which are useful in the present invention.

In a particular embodiment, the perfume oil comprises at least 40 wt. %, preferably at least 50 wt. %, more preferably at least 60 wt. % of ingredients having a log P above 3, preferably above 3.5 and even more preferably above 3.75.

Preferably, the perfume oil contains less than 10 wt. % of its own weight of primary alcohols, less than 15 wt. % of its own weight of secondary alcohols and less than 20% of its own weight of tertiary alcohols. Advantageously, the perfume used in the invention does not contain any primary alcohols and contains less than 15 wt. % of secondary and tertiary alcohols.

According to a particular embodiment, the perfume comprises at least 20 wt. %, preferably at least 25 wt. %, more preferably at least 40 wt. % of Bulky materials of groups 1 to 6, preferably 3 to 6.

The term Bulky materials is herein understood as perfuming ingredients having a high steric hindrance, i.e. having a substitution pattern which provides high steric hindrance and thus the Bulky materials are in particular those from one of the following groups:
Group 1: perfuming ingredients comprising a cyclohexane, cyclohexene, cyclohexanone or cyclohexenone ring substituted with at least one 1 to 4 nodes comprising substituent, preferably at least one linear or branched C₁ to C₄ alkyl or alkenyl substituent;
Group 2: perfuming ingredients comprising a cyclopentane, cyclopentene, cyclopentanone or cyclopentenone ring substituted with at least one 4 or more nodes comprising substituent, preferably at least one linear or branched C4 or longer, preferably C₄ to C₈ alkyl or alkenyl substituent;
Group 3: perfuming ingredients comprising a phenyl ring or perfuming ingredients comprising a cyclohexane, cyclohexene, cyclohexanone or cyclohexenone ring substituted with at least one 5 or more nodes comprising substituent, preferably at least one linear or branched C5 or longer, preferably C5 to C8, alkyl or alkenyl substituent, or with at least one phenyl substituent and optionally one or more 1 to 3 nodes comprising substituents, preferably one or more linear or branched C1 to C3 alkyl or alkenyl substituents;
Group 4: perfuming ingredients comprising at least two fused or linked 5 membered or 6 membered rings, preferably at least two fused or linked C5 and/or C6 rings;
Group 5: perfuming ingredients comprising a camphor-like ring structure, i.e. two 5 or 6 membered rings that are fused in a bridge-type fashion;
Group 6: perfuming ingredients comprising at least one 7 to 20 membered ring, preferably at least one C7 or C20 ring structure.

The term nodes as understood in this context means any atom which is able to provide at least two, preferably at least 3, more preferably 4, bonds to further atoms. Particular examples of nodes as herein understood are carbon atoms (up to 4 bonds to further atoms), nitrogen atoms (up to 3 bonds to further atoms), oxygen atoms (up to 2 bonds to further atoms) and sulfur (up to 2 bonds to further atoms). Particular examples of further atoms as understood in this context could be carbon atoms, nitrogen atoms, sulfur atoms, oxygen atoms and hydrogen atoms.

Examples of ingredients from each of these groups are:
Group 1: 2,4-dimethyl-3-cyclohexene-1-carbaldehyde (origin: Firmenich SA, Geneva, Switzerland), isocyclocitral, menthone, isomenthone, methyl 2,2-dimethyl-6-methylene-1-cyclohexanecarboxylate (origin: Firmenich SA, Geneva, Switzerland), nerone, terpineol, dihydroterpineol, terpenyl acetate, dihydroterpenyl acetate, dipentene, eucalyptol, hexylate, rose oxide, (S)-1,8-p-menthadiene-7-ol (origin: Firmenich SA, Geneva, Switzerland), 1-p-menthene-4-ol, (1RS,3RS,4SR)-3-p-mentanyl acetate, (1R,2S,4R)-4,6,6-trimethyl-bicyclo[3,1,1]heptan-2-ol, tetrahydro-4-methyl-2-phenyl-2H-pyran (origin: Firmenich SA, Geneva, Switzerland), cyclohexyl acetate, cyclanol acetate, 1,4-cyclohexane diethyldicarboxylate (origin: Firmenich SA, Geneva, Switzerland), (3ARS,6SR,7ASR)-perhydro-3,6-dimethyl-benzo[B]furan-2-one (origin: Firmenich SA, Geneva, Switzerland), ((6R)-perhydro-3,6-dimethyl-benzo[B]furan-2-one (origin: Firmenich SA, Geneva, Switzerland), 2,4,6-trimethyl-4-phenyl-1,3-dioxane, 2,4,6-trimethyl-3-cyclohexene-1-carbaldehyde;
Group 2: (E)-3-methyl-5-(2,2,3-trimethyl-3-cyclopenten-1-yl)-4-penten-2-ol (origin: Givaudan SA, Vernier, Switzerland), (1′R,E)-2-ethyl-4-(2′,2′,3′-trimethyl-3′-cyclopenten-1′-yl)-2-buten-1-ol (origin: Firmenich SA, Geneva, Switzerland), (1′R,E)-3,3-dimethyl-5-(2′,2′,3′-trimethyl-3′-cyclopenten-1′-yl)-4-penten-2-ol (origin: Firmenich SA, Geneva, Switzerland), 2-heptylcyclopentanone, methyl-cis-3-oxo-2-pentyl-1-cyclopentane acetate (origin: Firmenich SA, Geneva, Switzerland), 2,2,5-Trimethyl-5-pentyl-1-cyclopentanone (origin: Firmenich SA, Geneva, Switzerland), 3,3-dimethyl-5-(2,2,3-trimethyl-3-cyclopenten-1-yl)-4-penten-2-ol (origin: Firmenich SA, Geneva, Switzerland), 3-methyl-5-(2,2,3-trimethyl-3-cyclopenten-1-yl)-2-pentanol (origin, Givaudan SA, Vernier, Switzerland);
Group 3: damascones, 1-(5,5-dimethyl-1-cyclohexen-1-yl)-4-penten-1-one (origin: Firmenich SA, Geneva, Switzerland), nectalactone ((1′R)-2-[2-(4′-methyl-3′-cyclohexen-1′-yl)propyl]cyclopentanone), alpha-ionone, beta-ionone, damascenone, mixture of 1-(5,5-dimethyl-1-cyclohexen-1-yl)-4-penten-1-one and 1-(3,3-dimethyl-1-cyclohexen-1-yl)-4-penten-1-one (origin: Firmenich SA, Geneva, Switzerland), 1-(2,6,6-trimethyl-1-cyclohexen-1-yl)-2-buten-1-one (origin: Firmenich SA, Geneva, Switzerland), (1S,1′R)-[1-(3′,3′-Dimethyl-1′-cyclohexyl)ethoxycarbonyl]methyl propanoate (origin: Firmenich SA, Geneva, Switzerland), 2-tert-butyl-1-cyclohexyl acetate (origin: International Flavors and Fragrances, USA), 1-(2,2,3,6-tetramethyl-cyclohexyl)-3-hexanol (origin: Firmenich SA, Geneva, Switzerland), trans-1-(2,2,6-trimethyl-1-cyclohexyl)-3-hexanol (origin: Firmenich SA, Geneva, Switzerland), (E)-3-methyl-4-(2,6,6-trimethyl-2-cyclohexen-1-yl)-3-buten-2-one, terpenyl isobutyrate, 4-(1,1-dimethylethyl)-1-cyclohexyl acetate (origin: Firmenich SA, Geneva, Switzerland), 8-methoxy-1-p-menthene, (1S,1′R)-2-[1-(3′,3′-dimethyl-1′-cyclohexyl) ethoxy]-2-methylpropyl propanoate (origin: Firmenich SA, Geneva, Switzerland), para tert-butylcyclohexanone, menthenethiol, 1-methyl-4-(4-methyl-3-pentenyl)-3-cyclohexene-1-carbaldehyde, allyl cyclohexylpropionate, cyclohexyl salicylate, 2-methoxy-4-methylphenyl methyl carbonate, ethyl 2-methoxy-4-methylphenyl carbonate, 4-ethyl-2-methoxyphenyl methyl carbonate;
Group 4: Methyl cedryl ketone (origin: International Flavors and Fragrances, USA), a mixture of (1RS,2SR,6RS,7RS,8SR)-tricyclo[5.2.1.0˜2,6˜]dec-3-en-8-yl 2-methylpropanoate and (1RS,2SR,6RS,7RS,8SR)-tricyclo[5.2.1.0˜2,6˜]dec-4-en-8-yl 2-methylpropanoate, vetyverol, vetyverone, 1-(octahydro-2,3,8,8-tetramethyl-2-naphtalenyl)-1-ethanone (origin: International Flavors and Fragrances, USA), (5RS,9RS,10SR)-2,6,9,10-tetramethyl-1-oxaspiro[4.5]deca-3,6-diene and the (5RS,9SR,10RS) isomer, 6-ethyl-2,10,10-trimethyl-1-oxaspiro[4.5]deca-3,6-diene, 1,2,3,5,6,7-hexahydro-1,1,2,3,3-pentamethyl-4-indenone (origin: International Flavors and Fragrances, USA), a mixture of 3-(3,3-dimethyl-5-indanyl)propanal and 3-(1,1-dimethyl-5-indanyl)propanal (origin: Firmenich SA, Geneva, Switzerland), 3′,4-dimethyl-tricyclo[6.2.1.0(2,7)]undec-4-ene-9-spiro-2′-oxirane (origin: Firmenich SA, Geneva, Switzerland), 9/10-ethyldiene-3-oxatricyclo[6.2.1.0(2,7)]undecane, (perhydro-5,5,8A-trimethyl-2-naphthalenyl acetate (origin: Firmenich SA, Geneva, Switzerland), octalynol, (dodecahydro-3a,6,6,9a-tetramethyl-naphtho[2,1-b]furan, origin: Firmenich SA, Geneva, Switzerland), tricyclo[5.2.1.0(2,6)]dec-3-en-8-yl acetate and tricyclo[5.2.1.0(2,6)]dec-4-en-8-yl acetate as well as tricyclo[5.2.1.0(2,6)]dec-3-en-8-yl propanoate and tricyclo[5.2.1.0(2,6)]dec-4-en-8-yl propanoate, (+)-(1S,2S,3S)-2,6,6-trimethyl-bicyclo[3.1.1]heptane-3-spiro-2′-cyclohexen-4′-one;
Group 5: camphor, borneol, isobornyl acetate, 8-isopropyl-6-methyl-bicyclo[2.2.2]oct-5-ene-2-carbaldehyde, pinene, camphene, 8-methoxycedrane, (8-methoxy-2,6,6,8-tetramethyl-tricyclo[5.3.1.0(1,5)]undecane (origin: Firmenich SA, Geneva, Switzerland), cedrene, cedrenol, cedrol, mixture of 9-ethylidene-3-oxatricyclo[6.2.1.0(2,7)]undecan-4-one and 10-ethylidene-3-oxatricyclo[6.2.1.0(2,7)]undecan-4-one (origin: Firmenich SA, Geneva, Switzerland), 3-methoxy-7,7-dimethyl-10-methylene-bicyclo[4.3.1]decane (origin: Firmenich SA, Geneva, Switzerland);
Group 6: (trimethyl-13-oxabicyclo-[10.1.0]-trideca-4,8-diene (origin: Firmenich SA, Geneva, Switzerland), Ambrettolide LG ((E)-9-hexadecen-16-olide, origin: Firmenich SA, Geneva, Switzerland), pentadecenolide (origin: Firmenich SA, Geneva, Switzerland), muscenone (3-methyl-(4/5)-cyclopentadecenone, origin: Firmenich SA, Geneva, Switzerland), 3-methylcyclopentadecanone (origin: Firmenich SA, Geneva, Switzerland), pentadecanolide (origin: Firmenich SA, Geneva, Switzerland), cyclopentadecanone (origin: Firmenich SA, Geneva, Switzerland), 1-ethoxyethoxy)cyclododecane (origin: Firmenich SA, Geneva, Switzerland), 1,4-dioxacycloheptadecane-5,17-dione, 4,8-cyclododecadien-1-one;
Group 7: (+−)-2-methyl-3-[4-(2-methyl-2-propanyl)phenyl]propanal (origin: Givaudan SA, Vernier, Switzerland), 2,2,2-trichloro-1-phenylethyl acetate.

Preferably, the perfume comprises at least 30%, preferably at least 50%, more preferably at least 60% of ingredients selected from Groups 1 to 7, as defined above. More preferably said perfume comprises at least 30%, preferably at least 50% of ingredients from Groups 3 to 7, as defined above. Most preferably said perfume comprises at least 30%, preferably at least 50% of ingredients from Groups 3, 4, 6 or 7, as defined above.

According to another preferred embodiment, the perfume comprises at least 30%, preferably at least 50%, more preferably at least 60% of ingredients having a log P above 3, preferably above 3.5 and even more preferably above 3.75.

Preferably, the perfume used in the invention contains less than 10% of its own weight of primary alcohols, less than 15% of its own weight of secondary alcohols and less than 20% of its own weight of tertiary alcohols. Advantageously, the perfume used in the invention does not contain any primary alcohols and contains less than 15% of secondary and tertiary alcohols.

According to an embodiment, the oil phase (or the oil-based core) comprises:
25-100 wt % of a perfume oil comprising at least 15 wt % of high impact perfume raw materials having a Log T<−4, and
0-75 wt % of a density balancing material having a density greater than 1.07 g/cm³.

“High impact perfume raw materials” should be understood as perfume raw materials having a Log T<−4. The odor threshold concentration of a chemical compound is determined in part by its shape, polarity, partial charges and molecular mass. For convenience, the threshold concentration is presented as the common logarithm of the threshold concentration, i.e., Log [Threshold](“Log T”).

A “density balancing material” should be understood as a material having a density preferably greater than 1.07 g/cm3 and having preferably low or no odor.

The density of a component is defined as the ratio between its mass and its volume (g/cm3).

Several methods are available to determine the density of a component.

One may refer for example to the ISO 298:1998 method to measure d20 densities of essential oils.

The odor threshold concentration of a perfuming compound is determined by using a gas chromatograph (“GC”). Specifically, the gas chromatograph is calibrated to determine the exact volume of the perfume oil ingredient injected by the syringe, the precise split ratio, and the hydrocarbon response using a hydrocarbon standard of known concentration and chain-length distribution. The air flow rate is accurately measured and, assuming the duration of a human inhalation to last 12 seconds, the sampled volume is calculated. Since the precise concentration at the detector at any point in time is known, the mass per volume inhaled is known and hence the concentration of the perfuming compound. To determine the threshold concentration, solutions are delivered to the sniff port at the back-calculated concentration. A panelist sniffs the GC effluent and identifies the retention time when odor is noticed. The average across all panelists determines the odor threshold concentration of the perfuming compound. The determination of odor threshold is described in more detail in C. Vuilleumier et al., Multidimensional Visualization of Physical and Perceptual Data Leading to a Creative Approach in Fragrance Development, Perfume & Flavorist, Vol. 33, September, 2008, pages 54-61.

According to an embodiment, the high impact perfume raw materials having a Log T<−4 are selected from the group consisting of (+−)-1-methoxy-3-hexanethiol, 4-(4-hydroxy-1-phenyl)-2-butanone, 2-methoxy-4-(1-propenyl)-1-phenyl acetate, pyrazobutyle, 3-propylphenol, 1-(3-methyl-1-benzofuran-2-yl)ethanone, 2-(3-phenylpropyl)pyridine, 1-(3,3/5,5-dimethyl-1-cyclohexen-1-yl)-4-penten-1-one, 1-(5,5-dimethyl-1-cyclohexen-1-yl)-4-penten-1-one, a mixture comprising (3RS,3aRS,6SR,7ASR)-perhydro-3,6-dimethyl-benzo[b]furan-2-one and (3SR,3aRS,6SR,7ASR)-perhydro-3,6-dimethyl-benzo[b]furan-2-one, (+−)-1-(5-ethyl-5-methyl-1-cyclohexen-1-yl)-4-penten-1-one, (1'S,3′R)-1-methyl-2-[(1′,2′,2′-trimethylbicyclo[3.1.0]hex-3′-yl)methyl]cyclopropyl}methanol, (+−)-3-mercaptohexyl acetate, (2E)-1-(2,6,6-trimethyl-1,3-cyclohexadien-1-yl)-2-buten-1-one, H-methyl-2 h-1,5-benzodioxepin-3(4H)-one, (2E,6Z)-2,6-nonadien-1-ol, (4Z)-4-dodecenal, (+−)-4-hydroxy-2,5-dimethyl-3(2H)-furanone, methyl 2,4-dihydroxy-3,6-dimethylbenzoate, 3-methylindole, (+−)-perhydro-4alpha,8abeta-dimethyl-4a-naphthalenol, patchoulol, 2-methoxy-4-(1-propenyl)phenol, mixture comprising (+−)-5,6-dihydro-4-methyl-2-phenyl-2H-pyran and tetrahydro-4-methylene-2-phenyl-2H-pyran, mixture comprising 4-methylene-2-phenyltetrahydro-2H-pyran and (+−)-4-methyl-2-phenyl-3,6-dihydro-2H-pyran, 4-hydroxy-3-methoxybenzaldehyde, nonylenic aldehyde, 2-methoxy-4-propylphenol, 3-methyl-5-phenyl-2-pentenenitrile, 1-(spiro[4.5]dec-6/7-en-7-yl)-4-penten-1-one, 2-methoxynaphthalene, (−)-(3aR,5AS,9AS,9BR)-3a,6,6,9a-tetramethyldodecahydronaphtho[2,1-b]furan, 5-nonanolide, (3aR,5AS,9AS,9BR)-3a,6,6,9a-tetramethyldodecahydronaphtho[2,1-b]furan, 7-isopropyl-2H,4H-1,5-benzodioxepin-3-one, coumarin, 4-methylphenyl isobutyrate, (2E)-1-(2,6,6-trimethyl-1,3-cyclohexadien-1-yl)-2-buten-1-one, beta,2,2,3-tetramethyl-delta-methylene-3-cyclopentene-1-butanol, delta damascone ((2E)-1-[(1RS,2SR)-2,6,6-trimethyl-3-cyclohexen-1-yl]-2-buten-1-one), (+−)-3,6-dihydro-4,6-dimethyl-2-phenyl-2 h-pyran, anisaldehyde, paracresol, 3-ethoxy-4-hydroxybenzaldehyde, methyl 2-aminobenzoate, ethyl methylphenylglycidate, octalactone gamma, ethyl 3-phenyl-2-propenoate, (−)-(2E)-2-ethyl-4-[(1R)-2,2,3-trimethyl-3-cyclopenten-1-yl]-2-buten-1-ol, paracresyl acetate, dodecalactone, tricyclone, (+)-(3R,5Z)-3-methyl-5-cyclopentadecen-1-one, undecalactone, (1R,4R)-8-mercapto-3-p-menthanone, (3S,3AS,6R,7AR)-3,6-dimethylhexahydro-1-benzofuran-2(3H)-one, beta ionone, (+−)-6-pentyltetrahydro-2H-pyran-2-one, (3E,5Z)-1,3,5-undecatriene, 10-undecenal, (9E)-9-undecenal (9Z)-9-undecenal, (Z)-4-decenal, (+−)-ethyl 2-methylpentanoate, 1,2-diallyldisulfane, 2-tridecenenitrile, 3-tridecenenitrile, (+−)-2-ethyl-4,4-dimethyl-1,3-oxathiane, (+)-(3R,5Z)-3-methyl-5-cyclopentadecen-1-one, 3-(4-tert-butylphenyl)propanal, allyl (cyclohexyloxy)acetate, methylnaphthylketone, (+−)-(4E)-3-methyl-4-cyclopentadecen-1-one, (+−)-5E3-methyl-5-cyclopentadecen-1-one, cyclopropylmethyl 3-hexenoate, (4E)-4-methyl-5-(4-methylphenyl)-4-pentenal, (+−)-1-(5-propyl-1,3-benzodioxol-2-yl)ethanone, 4-methyl-2-pentylpyridine, (+−)-(E)-3-methyl-4-(2,6,6-trimethyl-2-cyclohexen-1-yl)-3-buten-2-one, (3aRS,5aSR,9aSR,9bRS)-3a,6,6,9a-tetramethyldodecahydronaphtho[2,1-b]furan, (2S,5R)-5-methyl-2-(2-propanyl)cyclohexanone oxime, 6-hexyltetrahydro-2H-pyran-2-one, (+−)-3-(3-isopropyl-1-phenyl)butanal, methyl 2-(3-oxo-2-pentylcyclopentyl)acetate, 1-(2,6,6-trimethyl-1-cyclohex-2-enyl)pent-1-en-3-one, indol, 7-propyl-2H,4H-1,5-benzodioxepin-3-one, ethyl praline, (4-methylphenoxy)acetaldehyde, ethyl tricyclo[5.2.1.0.2,6]decane-2-carboxylate, (+)-(1'S,2S,E)-3,3-dimethyl-5-(2′,2′,3′-trimethyl-3′-cyclopenten-1′-yl)-4-penten-2-ol, (4E)-3,3-dimethyl-5-[(1R)-2,2,3-trimethyl-3-cyclopenten-1-yl]-4-penten-2-ol, 8-isopropyl-6-methyl-bicyclo[2.2.2]oct-5-ene-2-carbaldehyde, methylnonylacetaldehyde, 4-formyl-2-methoxyphenyl 2-methylpropanoate, (E)-4-decenal, (+−)-2-ethyl-4-(2,2,3-trimethyl-3-cyclopenten-1-yl)-2-buten-1-ol, (1R,5R)-4,7,7-trimethyl-6-thiabicyclo[3.2.1]oct-3-ene, (1R,4R,5R)-4,7,7-trimethyl-6-thiabicyclo[3.2.1]octane, (−)-(3R)-3,7-dimethyl-1,6-octadien-3-ol, (E)-3-phenyl-2-propenenitrile, 4-methoxybenzyl acetate, (E)-3-methyl-5-(2,2,3-trimethyl-3-cyclopenten-1-yl)-4-penten-2-ol, allyl (2/3-methylbutoxy)acetate, (+−)-(2E)-1-(2,6,6-trimethyl-2-cyclohexen-1-yl)-2-buten-1-one, (1E)-1-(2,6,6-trimethyl-1-cyclohexen-1-yl)-1-penten-3-one, and mixtures thereof.

According to an embodiment, perfume raw materials having a Log T<−4 are chosen in the group consisting of aldehydes, ketones, alcohols, phenols, esters lactones, ethers, epoxydes, nitriles and mixtures thereof.

According to an embodiment, perfume raw materials having a Log T<−4 comprise at least one compound chosen in the group consisting of alcohols, phenols, esters lactones, ethers, epoxydes, nitriles and mixtures thereof, preferably in amount comprised between 20 and 70 wt. % based on the total weight of the perfume raw materials having a Log T<−4.

According to an embodiment, perfume raw materials having a Log T<−4 comprise between 20 and 70 wt. % by weight of aldehydes, ketones, and mixtures thereof based on the total weight of the perfume raw materials having a Log T<−4.

The remaining perfume raw materials contained in the oil-based core may have therefore a Log T>−4.

According to an embodiment, the perfume raw materials having a Log T>−4 are chosen in the group consisting of ethyl 2-methylbutyrate, (E)-3-phenyl-2-propenyl acetate, (+−)-6/8-sec-butylquinoline, (+−)-3-(1,3-benzodioxol-5-yl)-2-methylpropanal, verdyl propionate, 1-(octahydro-2,3,8,8-tetramethyl-2-naphtalenyl)-1-ethanone, methyl 2-((1RS,2RS)-3-oxo-2-pentylcyclopentyl)acetate, (+−)-(E)-4-methyl-3-decen-5-ol, 2,4-dimethyl-3-cyclohexene-1-carbaldehyde, 1,3,3-trimethyl-2-oxabicyclo[2.2.2]octane, tetrahydro-4-methyl-2-(2-methyl-1-propenyl)-2H-pyran, dodecanal, 1-oxa-12/13-cyclohexadecen-2-one, (+−)-3-(4-isopropylphenyl)-2-methylpropanal, aldehyde C11, (+−)-2,6-dimethyl-7-octen-2-ol, allyl 3-cyclohexylpropanoate, (Z)-3-hexenyl acetate, 5-methyl-2-(2-propanyl)cyclohexanone, allyl heptanoate, 2-(2-methyl-2-propanyl)cyclohexyl acetate, 1,1-dimethyl-2-phenylethyl butyrate, geranyl acetate, neryl acetate, (+−)-1-phenylethyl acetate, 1,1-dimethyl-2-phenylethyl acetate, 3-methyl-2-butenyl acetate, ethyl 3-oxobutanoate, (2Z)-ethyl 3-hydroxy-2-butenoate, 8-p-menthanol, 8-p-menthanyl acetate, 1-p-menthanyl acetate, (+−)-2-(4-methyl-3-cyclohexen-1-yl)-2-propanyl acetate, (+−)-2-methylbutyl butanoate, 2-{(1S)-1-[(1R)-3,3-dimethylcyclohexyl]ethoxy}-2-oxoethyl propionate, 3,5,6-trimethyl-3-cyclohexene-1-carbaldehyde, 2,4,6-trimethyl-3-cyclohexene-1-carbaldehyde, 2-cyclohexylethyl acetate, octanal, ethyl butanoate, (+−)-(3E)-4-(2,6,6-trimethyl-1/2-cyclohexen-1-yl)-3-buten-2-one, 1-[(1RS,6SR)-2,2,6-trimethylcyclohexyl]-3-hexanol, 1,3,3-trimethyl-2-oxabicyclo[2.2.2]octane, 1,3,3-trimethyl-2-oxabicyclo[2.2.2]octane, ethyl hexanoate, undecanal, decanal, 2-phenylethyl acetate, (1S,2S,4S)-1,7,7-trimethylbicyclo[2.2.1]heptan-2-ol, (1S,2R,4S)-1,7,7-trimethylbicyclo[2.2.1]heptan-2-ol), (+−)-3,7-dimethyl-3-octanol, 1-methyl-4-(2-propanylidene)cyclohexene, (+)-(R)-4-(2-methoxypropan-2-yl)-1-methylcyclohex-1-ene, verdyl acetate, (3R)-1-[(1R,6S)-2,2,6-trimethylcyclohexyl]-3-hexanol, (3S)-1-[(1R,6S)-2,2,6-trimethylcyclohexyl]-3-hexanol, (3R)-1-[(1S,6S)-2,2,6-trimethylcyclohexyl]-3-hexanol, (+)-(1S,1′R)-2-[1-(3′,3′-dimethyl-1′-cyclohexyl)ethoxy]-2-methylpropyl propanoate, and mixtures thereof.

The nature of high impact perfume raw materials having a Log T<−4 and density balancing material having a density greater than 1.07 g/cm3 are described in WO2018115250, the content of which are included by reference.

The term “biocide” refers to a chemical substance capable of killing living organisms (e.g. microorganisms) or reducing or preventing their growth and/or accumulation. Biocides are commonly used in medicine, agriculture, forestry, and in industry where they prevent the fouling of, for example, water, agricultural products including seed, and oil pipelines. A biocide can be a pesticide, including a fungicide, herbicide, insecticide, algicide, molluscicide, miticide and rodenticide; and/or an antimicrobial such as a germicide, antibiotic, antibacterial, antiviral, antifungal, antiprotozoal and/or antiparasite.

As used herein, a “pest control agent” indicates a substance that serves to repel or attract pests, to decrease, inhibit or promote their growth, development or their activity. Pests refer to any living organism, whether animal, plant or fungus, which is invasive or troublesome to plants or animals, pests include insects notably arthropods, mites, spiders, fungi, weeds, bacteria and other microorganisms.

According to an embodiment, the perfume formulation comprises
0 to 60 wt. % of a hydrophobic solvent (based on the total weight of the perfume formulation),
40 to 100 wt. % of a perfume oil (based on the total weight of the perfume formulation), wherein the perfume oil has at least two, preferably all of the following characteristics:
at least 35%, preferably at least 40%, preferably at least 50%, more preferably at least 60% of perfuming ingredients having a log P above 3, preferably above 3.5,
at least 20%, preferably at least 25%, preferably at least 30%, more preferably at least 40% of Bulky materials of groups 1 to 6, preferably 3 to 6 as previously defined and
at least 15%, preferably at least 20%, more preferably at least 25%, even more preferably at least 30% of high impact perfume materials having a Log T<−4 as previously defined,
optionally, further hydrophobic active ingredients.

According to a particular embodiment, the perfume comprises 0 to 60 wt. % of a hydrophobic solvent.

According to a particular embodiment, the hydrophobic solvent is a density balancing material preferably chosen in the group consisting of benzyl salicylate, benzyl benzoate, cyclohexyl salicylate, benzyl phenylacetate, phenylethyl phenylacetate, triacetin, ethyl citrate, methyl and ethyl salicylate, benzyl cinnamate, and mixtures thereof.

In a particular embodiment, the hydrophobic solvent has Hansen Solubility Parameters compatible with entrapped perfume oil.

The term “Hansen solubility parameter” is understood refers to a solubility parameter approach proposed by Charles Hansen used to predict polymer solubility and was developed around the basis that the total energy of vaporization of a liquid consists of several individual parts. To calculate the “weighted Hansen solubility parameter” one must combine the effects of (atomic) dispersion forces, (molecular) permanent dipole-permanent dipole forces, and (molecular) hydrogen bonding (electron exchange). The weighted Hansen solubility parameter” is calculated as (δD2+δP2+δH2)0.5, wherein δD is the Hansen dispersion value (also referred to in the following as the atomic dispersion fore), δP is the Hansen polarizability value (also referred to in the following as the dipole moment), and δH is the Hansen Hydrogen-bonding (“h-bonding”) value (also referred to in the following as hydrogen bonding). For a more detailed description of the parameters and values, see Charles Hansen, The Three Dimensional Solubility Parameter and Solvent Diffusion Coefficient, Danish Technical Press (Copenhagen, 1967).

Euclidean difference in solubility parameter between a fragrance and a solvent is calculated as (4*(δDsolvent−δDfragrance)2+(δPsolvent−δPfragrance)2+(δHsolvent−δHfragrance)2)0.5, in which δDsolvent, δPsolvent, and δHsolvent, are the Hansen dispersion value, Hansen polarizability value, and Hansen h-bonding values of the solvent, respectively; and δDfragrance, δPfragrance, and Hfragrance are the Hansen dispersion value, Hansen polarizability value, and Hansen h-bonding values of the fragrance, respectively.

In a particular embodiment, the perfume oil and the hydrophobic solvent have at least two Hansen solubility parameters selected from a first group consisting of: an atomic dispersion force (δD) from 12 to 20, a dipole moment (δP) from 1 to 8, and a hydrogen bonding (δH) from 2.5 to 11.

In a particular embodiment, the perfume oil and the hydrophobic solvent have at least two Hansen solubility parameters selected from a second group consisting of: an atomic dispersion force (δD) from 12 to 20, preferably from 14 to 20, a dipole moment (δP) from 1 to 8, preferably from 1 to 7, and a hydrogen bonding (δH) from 2.5 to 11, preferably from 4 to 11

According to a particular embodiment, the hydrophobic material is free of any active ingredient (such as perfume). According to this particular embodiment, it comprises, preferably consists of hydrophobic solvents, preferably chosen in the group consisting of isopropyl myristate, tryglycerides (e.g. Neobee® MCT oil, vegetable oils), D-limonene, silicone oil, mineral oil, and mixtures thereof with optionally hydrophilic solvents preferably chosen in the group consisting of 1,4-butanediol, benzyl alcohol, triethyl citrate, triacetin, benzyl acetate, ethyl acetate, propylene glycol (1,2-propanediol), 1,3-propanediol, dipropylene glycol, glycerol, glycol ethers and mixtures thereof.

### Acyl Chloride

According to a particular embodiment, the acyl chloride has the following formula (I)

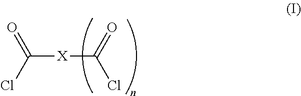

wherein n is an integer varying between 1 and 8, preferably between 1 and 6, more preferably between 1 and 4, and
wherein X is either an (n+1)-valent C₂ to C₄₅ hydrocarbon group optionally comprising at least one group selected from (i) to (xi),

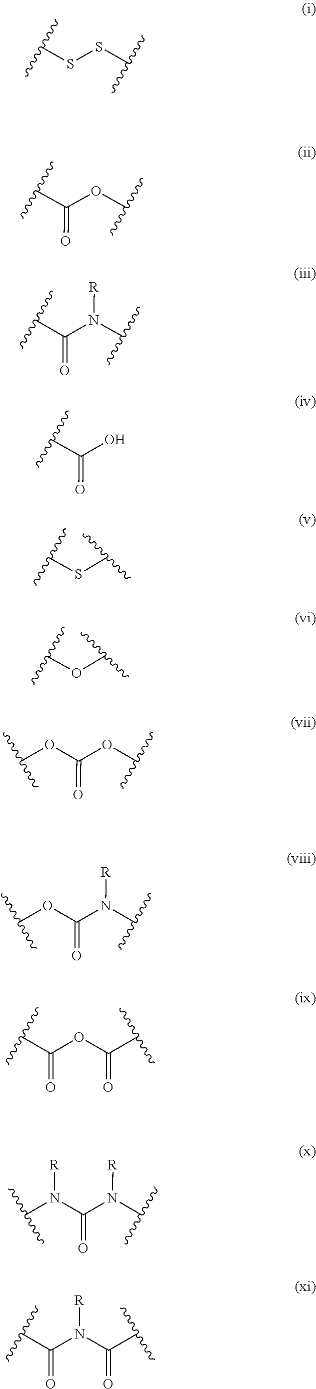

wherein R is a hydrogen atom or an alkyl group such as a methyl or an ethyl group, preferably a hydrogen atom.

It is understood that by “ . . . hydrocarbon group . . . ” it is meant that said group consists of hydrogen and carbon atoms and can be in the form of an aliphatic hydrocarbon, i.e. linear or branched saturated hydrocarbon (e.g. alkyl group), a linear or branched unsaturated hydrocarbon (e.g. alkenyl or alkynil group), a saturated cyclic hydrocarbon (e.g. cycloalkyl) or an unsaturated cyclic hydrocarbon (e.g. cycloalkenyl or cycloalkynyl), or can be in the form of an aromatic hydrocarbon, i.e. aryl group, or can also be in the form of a mixture of said type of groups, e.g. a specific group may comprise a linear alkyl, a branched alkenyl (e.g. having one or more carbon-carbon double bonds), a (poly)cycloalkyl and an aryl moiety, unless a specific limitation to only one type is mentioned. Similarly, in all the embodiments of the invention, when a group is mentioned as being in the form of more than one type of topology (e.g. linear, cyclic or branched) and/or being saturated or unsaturated (e.g. alkyl, aromatic or alkenyl), it is also meant a group which may comprise moieties having any one of said topologies or being saturated or unsaturated, as explained above. Similarly, in all the embodiments of the invention, when a group is mentioned as being in the form of one type of saturation or unsaturation, (e.g. alkyl), it is meant that said group can be in any type of topology (e.g. linear, cyclic or branched) or having several moieties with various topologies. It is understood that with the term “ . . . a hydrocarbon group, optionally comprising . . . ” it is meant that said hydrocarbon group optionally comprises heteroatoms to form ether, thioether, amine, nitrile or carboxylic acid groups and derivatives (including for example esters, acids, amide). These groups can either substitute a hydrogen atom of the hydrocarbon group and thus be laterally attached to said hydrocarbon, or substitute a carbon atom (if chemically possible) of the hydrocarbon group and thus be inserted into the hydrocarbon chain or ring.

According to an embodiment, the acyl chloride is chosen in the group consisting of diacyl chloride, triacyl chloride and mixtures thereof.

According to a particular embodiment, the acyl chloride is chosen from the group consisting of benzene-1,3,5-tricarbonyl trichloride (trimesoyl trichloride), benzene-1,2,4-tricarbonyl trichloride, benzene-1,2,4-tricarbonyl trichloride, benzene-1,2,4,5-tetracarbonyl tetrachloride, cyclohexane-1,3,5-tricarbonyl trichloride, propane-1,2,3-tricarbonyl trichloride, cyclohexane-1,2,4,5-tetracarbonyl tetrachloride, 2,2′-disulfanediyldisuccinyl dichloride, 2-(2-chloro-2-oxo-ethyl)sulfanylbutanedioyl dichloride, (4-chloro-4-oxobutanoyl)-L-glutamoyl dichloride, (S)-4-((1,5-dichloro-1,5-dioxopentan-2-yl)amino)-4-oxobutanoic acid, 2,2-bis[(4-chloro-4-oxo-butanoyl)oxymethyl]butyl 4-chloro-4-oxo-butanoate, [2-[2,2-bis[(4-chloro-4-oxo-butanoyl)oxymethyl]butoxymethyl]-2-[(4-chloro-4-oxo-butanoyl)oxymethyl]butyl]4-chloro-4-oxo-butanoate, 2,2-bis[(2-chlorocarbonylbenzoyl)oxymethyl]butyl 2-chlorocarbonyl-benzoate, [2-[2,2-bis[(2-chlorocarbonylbenzoyl)oxymethyl]butoxymethyl]-2-[(2-chlorocarbonylbenzoyl)oxymethyl]butyl]2-chlorocarbonylbenzoate, 4-(2,4,5-trichlorocarbonylbenzoyl)oxybutyl 2,4,5-trichlorocarbonyl-benzoate, propane-1,2,3-triyl tris(4-chloro-4-oxobutanoate), propane-1,2-diyl bis(4-chloro-4-oxobutanoate) and mixtures thereof.

According to an embodiment, the acyl chloride is a diacyl chloride, preferably chosen in the group consisting of terephthaloyl chloride, diglycolyl dichloride, phthaloyl chloride, isophthaloyl chloride, adipoyl chloride, sebacoyl chloride, succinyl chloride, glutaryl chloride, pimeloyl chloride, dodecanedioyl dichloride, suberoyl chloride, azelaoyl chloride, malonyl chloride, fumaryl chloride, oxyalyl chloride, 4,4′-oxybis(benzoyl chloride), 2,2′-oxydiacetyl chloride, 4,4′-biphenyldicarbonyl chloride, azobenzene-4,4′-dicarbonyl dichloride, 2,5-furandicarbonyl dichloride, and mixtures thereof.

According to a particular embodiment, the acyl chloride is used in an amount comprised between 0.1 and 50%, preferably between 0.5 and 15% by weight based on the total weight of the core.

### Co-Oligopeptide

According to the invention, a “co-oligopeptide” comprises at least 2 types of amino acid units linked by peptide bonds. According to an embodiment, the “co-oligopeptide” comprises between 2 and 50 amino-acid units, more preferably between 2 and 30 amino acid units (also named residues) linked by peptide bonds.

According to an embodiment, a “co-oligopeptide” comprises at least 2 types of amino acid units and is defined as (a) molecule(s) consisting of at least 2 and less than 25 amino acid units (also named residues) linked by peptide bonds.

The co-oligopeptide according to the invention has an average molecular weight less than 4000 g/mol.

By “2 types of amino acid units”, it should be understood that the co-oligopeptide comprises at least two different amino acid units.

The wording “co-oligopeptide” or “co-oligopeptide mixture” can be used indifferently in the present invention. According to the invention, the co-oligopeptide mixture comprises at least one co-oligopeptide.

In other words, according to the invention, the wording “co-oligopeptide” or “co-oligopeptide mixture” can also encompass a mixture of co-oligopeptide, oligopeptide and optionally free amino acids.

“Amino acid” and “free amino acid” can be used indifferently in the present invention.

The average molecular weight can be easily determined by a skilled person in the art.

According to one embodiment, the average molecular weight of the co-oligopeptide can be extracted from the ¹H NMR analysis, for example using a Bruker AV-300 spectrometer.

According to an embodiment, the co-oligopeptide has an average molecular weight less than 10 000 g/mol, preferably less than 4000 g/mol, preferably up to 2000 g/mol, more preferably between 200 to 1000 g/mol, even more preferably between 300 to 1000 g/mol.

According to an embodiment, the co-oligopeptide has an average molecular weight between 200 and less than 10 000 g/mol, preferably between 200 and less than 4000 g/mol. According to an embodiment, the co-oligopeptide has an average molecular weight between 200 and 2000 g/mol. According to an embodiment, the co-oligopeptide has an average molecular weight between 200 and 1000 g/mol. According to an embodiment, the co-oligopeptide has an average molecular weight between 300 and less than 10 000 g/mol, preferably between 300 and less than 4000 g/mol. According to an embodiment, the co-oligopeptide has an average molecular weight between 300 and 2000 g/mol. According to an embodiment, the co-oligopeptide has an average molecular weight between 300 and 1000 g/mol.

An “oligopeptide” as defined in the present invention is a homo-oligopeptide which comprises only one type of repeating amino acid unit linked by peptide bonds. According to an embodiment, it comprises between 2 and 30, preferably between 2 and 25 amino acid units (also named residues) linked by peptide bonds.

According to an embodiment, an “oligopeptide” is a homo-oligopeptide which comprises only one type of repeating amino acid unit and is defined as (a) molecule(s) consisting of at least 2 and less than 25 amino acid units (also named residues) linked by peptide bonds.
It should be understood that the oligopeptide has an average molecular weight less than 4000 g/mol.
   It should also be understood that the oligopeptide is a homo-oligopeptide which comprises only one type of repeating amino acid unit, contrary to the co-oligopeptide which comprises at least 2 type of amino acid units.

“Oligopeptide” and “peptide” can be used indifferently in the present invention.

According to an embodiment, the co-oligopeptide can be linear, branched, or random.

According to an embodiment, the co-oligopeptide comprises at least 2 amino groups, preferably at least 3 amino groups.

According to an embodiment, the co-oligopeptide comprises at least two different amino acids chosen from the group consisting of lysine, arginine, ornithine, histidine, tryptophan, aspartic acid, glutamic acid, serine, threonine, asparagine, glutamine, glycine, proline, selenocysteine, alanine, valine, isoleucine, leucine, methionine, phenylalanine, tyrosine, citrulline, cysteine and mixtures thereof.

According to an embodiment, the co-oligopeptide comprises at least two different amino acids A and B, wherein the amino acid A is chosen from the group consisting of lysine, arginine, ornithine, histidine, tryptophan and mixtures thereof, and wherein the amino acid B is chosen from the group consisting of lysine, arginine, ornithine, histidine, tryptophan, aspartic acid, glutamic acid, serine, threonine, asparagine, glutamine, glycine, proline, selenocysteine, alanine, valine, isoleucine, leucine, methionine, phenylalanine, tyrosine, citrulline, cysteine and mixtures thereof.

According to an embodiment, the amino acid A is lysine.

Preferably, the amino acid B is chosen from the group consisting of arginine, histidine, serine, threonine, glycine, alanine, valine, isoleucine, leucine, tyrosine.

“Amino acid A” and “amino acid unit A” can be used indifferently.

“Amino acid B” and “amino acid unit B” can be used indifferently.

According to an embodiment, the co-oligopeptide mixture comprises a co-oligopeptide and an oligopeptide as defined previously and optionally a free amino acid.

According to an embodiment, the co-oligopeptide is present in an amount of at least 15%, preferably at least 30% by weight based on the total weight of the co-oligopeptide mixture.

According to an embodiment, the co-oligopeptide is present in an amount comprised between 15% and 100%, preferably between 30% and 80% by weight based on the total weight of the co-oligopeptide mixture.

According to an embodiment, when present, the oligopeptide is present in an amount of at least 5%, preferably at least 10% by weight based on the total weight of the co-oligopeptide mixture.

According to an embodiment, when present, the oligopeptide is present in an amount comprised between 5% and 85%, preferably between 10% and 50% by weight based on the total weight of the co-oligopeptide mixture.

According to an embodiment, when present, the free amino acid is present in an amount of at least 5%, preferably at least 10% by weight based on the total weight of the co-oligopeptide mixture.

According to an embodiment, when present, the free amino acid is present in an amount comprised between 5% and 50%, preferably between 10% and 20% by weight based on the total weight of the co-oligopeptide mixture.

Free amino acid can be lysine, arginine, histidine, tryptophan, ornithine, glutamine, asparagine, citrulline, and mixtures thereof.

According to an embodiment the co-oligopeptide mixture is a mixture of hydrolysed protein containing amino acid units chosen in the list of lysine, arginine, histine, tryptophan, ornithine, citrulline, glutamine, or asparagine and other amino acid units.

According to an embodiment the co-oligopeptide does not contain free amino acid.

When present, the oligopeptide is preferably chosen in the group consisting of oligo-lysine, oligo-arginine, oligo-histidine, oligo-tryptophan, oligo-serine, oligo-glutamine, oligo-threonine, oligo-asparagine, oligo-ornithine, oligo-citrulline.

According to an embodiment, the oligopeptide is chosen in the group consisting of oligo-lysine, oligo-arginine, oligo-histidine, oligo-tryptophan, oligo-ornithine.

According to an embodiment, the oligopeptide is oligo-lysine, more specifically linear oligo-lysine or (hyper)branched oligo-lysine According to a particular embodiment, oligo-lysine can be α-oligo-lysine or ε-oligo-lysine.

According to a particular embodiment, oligo-lysine can be oligo-L-lysine, more specifically α-oligo-L-lysine or ε-oligo-L-lysine; oligo-D-lysine, more specifically, α-oligo-D-lysine or ε-oligo-D-lysine; oligo-D, L-lysine, more specifically, α-oligo-D, L-lysine or ε-oligo-D, L-lysine, and mixtures thereof.

According to an embodiment, the co-oligopeptide mixture does not contain oligopeptide.

According to an embodiment, the co-oligopeptide can be used to react with the acyl chloride.

According to a particular embodiment, the polyamide shell comprises the reaction product between the co-oligopeptide and at least one diacyl chloride.

According to a particular embodiment, the polyamide shell comprises the reaction product between the co-oligopeptide and phthaloyl chloride.

According to a particular embodiment, the polyamide shell comprises the reaction product between the co-oligopeptide and isophthaloyl chloride.

According to a particular embodiment, the polyamide shell comprises the reaction product between the co-oligopeptide and terephthaloyl chloride.

(tere/iso)phthaloyl chloride means terephthaloyl chloride or isophthaloyl chloride or phthaloyl chloride.

According to an embodiment, the mole ratio of amino group on average from co-oligopeptide to acyl chloride group from the acyl chloride is 0.05:1 to 65:1, preferably 0.1:1 to 10:1.

Co-oligopeptide used in the present invention can be commercially available or can be prepared.

Co-oligopeptide can be prepared by an enzymatic synthesis, more particularly comprising the steps of:
(i) Preparing an amino acid ester A aqueous solution,
(ii) Adding an enzyme solution to the solution of step (i) to obtain a mixture
(iii) Stirring the mixture, preferably under heating
wherein an amino acid ester B, different from the amino acid ester A, is added in step (i) or after step (iii).

When the amino acid ester B is added after step (iii), an additional enzyme solution can be added and a further stirring step is performed, preferably under heating to obtain the co-oligopeptide.

The amino acid ester A can be lysine alkyl ester, arginine alkyl ester, ornithine alkyl ester, histidine alkyl ester, tryptophan alkyl ester.

The amino acid ester B can be lysine alkyl ester, arginine alkyl ester, ornithine alkyl ester, histidine alkyl ester, tryptophan alkyl ester, aspartic acid alkyl ester, glutamic acid alkyl ester, serine alkyl ester, threonine alkyl ester, asparagine alkyl ester, glutamine alkyl ester, glycine alkyl ester, proline alkyl ester, selenocysteine alkyl ester, alanine alkyl ester, valine alkyl ester, isoleucine alkyl ester, leucine alkyl ester, methionine alkyl ester, phenylalanine alkyl ester, tyrosine alkyl ester, citrulline alkyl ester, cysteine alkyl ester.

The enzyme may be chosen from the list consisting of bromelain, papain, ficin, actinidin, zingibain, legumain, cardosins A or B, enzyme from germinated plant seeds, such as leek, red clover, broccoli, enzyme from asparagus, enzyme from onion, chymotrypsin, trypsin, carboxypeptidase, pepsin, cathepsin, calpains, chymosin, thrombin, serine endopeptidase (Alcalase®), a bacterial enzyme produced by a selected strain of Bacillus amyloliquefaciens (Neutrase®), mixture of enzyme isolated from the extracellular fluid of Streptomyces griseus (Pronase®), serine endopeptidase derived from Bacillus amyloliquefaciens and manufactured in recombinant B. subtilis (Purafect Prime® L), alkaline enzyme, metallo enzyme, microbial enzyme, and mixtures thereof.

### Stabilizer

According to an embodiment, the polyamide shell comprising the reaction product between at least one acyl chloride and at least one co-oligopeptide in the presence of a stabilizer.

According to the nature of the stabilizer, the stabilizer can be part of the shell. It may react with the acyl chloride and the co-oligopeptide.
Therefore, according to an embodiment, the polyamide shell comprises the reaction product between at least one acyl chloride, at least one co-oligopeptide and at least one stabilizer.

According to an embodiment, the polyamide shell comprises the reaction product between:
at least one acyl chloride,
at least one co-oligopeptide,
at least one stabilizer,
optionally at least one oligopeptide, and
optionally at least one free amino acid.

According to an embodiment, the microcapsule or the microcapsule slurry comprises a stabilizer.

The stabilizer is defined as below.

### Biodegradability

In a particular embodiment, the shell material is a biodegradable material.

In a particular embodiment, the shell has a biodegradability of at least 40%, preferably at least 45%, 50%, 60%, 65%, 70%, 75%, 80%, 85%, 90%, 95% or 98%, within 60 days according to OECD301F.

In a particular embodiment, the core-shell microcapsule has a biodegradability of at least 40%, preferably at least 60%, preferably at least 65%, 70%, 75%, 80%, 85%, 90%, 95% or 98% within 60 days according to OECD301F.

Thereby it is understood that the core-shell microcapsule including all components, such as the core, shell and optionally coating may have a biodegradability of at least 40%, preferably at least 60%, preferably at least 65%, 70%, 75%, 80%, 85%, 90%, 95% or 98% within 60 days according to OECD301F.

In a particular embodiment, the oil-based core, preferably perfume oil has a biodegradability of at least 40%, preferably at least 60%, preferably at least 65%, 70%, 75%, 80%, 85%, 90%, 95% or 98% within 60 days according to OECD301F.

OECD301F is a standard test method on the biodegradability from the Organization of Economic Co-operation and Development.

A typical method for extracting the shell for measuring the biodegradability is disclosed in Gasparini and all in Molecules 2020, 25,718.

### Optional Outer Coating

According to a particular embodiment of the invention, the microcapsule comprises an outer coating, wherein the outer coating comprises a coating material selected from the group consisting of a non-ionic polysaccharide, a cationic polymer, a polysuccinimide derivative (as described for instance in WO2021185724) and mixtures thereof to form an outer coating to the microcapsule.

Non-ionic polysaccharide polymers are well known to a person skilled in the art and are described for instance in WO2012/007438 page 29, lines 1 to 25 and in WO2013/026657 page 2, lines 12 to 19 and page 4, lines 3 to 12. Preferred non-ionic polysaccharides are selected from the group consisting of locust bean gum, xyloglucan, guar gum, hydroxypropyl guar, hydroxypropyl cellulose and hydroxypropyl methyl cellulose.

Cationic polymers are well known to a person skilled in the art. Preferred cationic polymers have cationic charge densities of at least 0.5 meq/g, more preferably at least about 1.5 meq/g, but also preferably less than about 7 meq/g, more preferably less than about 6.2 meq/g. The cationic charge density of the cationic polymers may be determined by the Kjeldahl method as described in the US Pharmacopoeia under chemical tests for Nitrogen determination. The preferred cationic polymers are chosen from those that contain units comprising primary, secondary, tertiary and/or quaternary amine groups that can either form part of the main polymer chain or can be borne by a side substituent directly connected thereto. The weight average (Mw) molecular weight of the cationic polymer is preferably between 10,000 and 3.5M Dalton, more preferably between 50,000 and 1.5M Dalton. According to a particular embodiment, one will use cationic polymers based on acrylamide, methacrylamide, N-vinylpyrrolidone, quaternized N,N-dimethylaminomethacrylate, diallyldimethylammonium chloride, quaternized vinylimidazole (3-methyl-1-vinyl-1H-imidazol-3-ium chloride), vinylpyrrolidone, acrylamidopropyltrimonium chloride, cassia hydroxypropyltrimonium chloride, guar hydroxypropyltrimonium chloride or polygalactomannan 2-hydroxypropyltrimethylammonium chloride ether, starch hydroxypropyltrimonium chloride and cellulose hydroxypropyltrimonium chloride. Preferably copolymers shall be selected from the group consisting of polyquaternium-5, polyquaternium-6, polyquaternium-7, polyquaternium10, polyquaternium-11, polyquaternium-16, polyquaternium-22, polyquaternium-28, polyquaternium-43, polyquaternium-44, polyquaternium-46, cassia hydroxypropyltrimonium chloride, guar hydroxypropyltrimonium chloride or polygalactomannan 2-hydroxypropyltrimethylammonium chloride ether, starch hydroxypropyltrimonium chloride and cellulose hydroxypropyltrimonium chloride. As specific examples of commercially available products, one may cite Salcare® SC60 (cationic copolymer of acrylamidopropyltrimonium chloride and acrylamide, origin: BASF) or Luviquat®, such as the PQ 11N, FC 550 or Style (polyquaternium-11 to 68 or quaternized copolymers of vinylpyrrolidone origin: BASF), or also the Jaguar® (C13S or C17, origin Rhodia).

According to any one of the above embodiments of the invention, there is added an amount of polymer described above comprised between about 0% and 5% w/w, or even between about 0.1% and 2% w/w, percentage being expressed on a w/w basis relative to the total weight of the slurry. It is clearly understood by a person skilled in the art that only part of said added polymers will be incorporated into/deposited on the microcapsule shell.

### Optional Components

When microcapsules are in the form of a slurry, the microcapsule slurry can comprise auxiliary ingredients selected from the group of thickening agents/rheology modifiers, antimicrobial agents, opacity-building agents, mica particles, salt, pH stabilizers/buffering ingredients, preferably in an amount comprised between 0 and 15% by weight based on the total weight of the slurry.

According to another embodiment, the microcapsule slurry of the invention comprises additional free (i.e non-encapsulated) perfume, preferably in an amount comprised between 5 and 50% by weight based on the total weight of the slurry.

In a particular embodiment, the core-shell microcapsules are isolated by drying the obtained core-shell microcapsule slurry. Drying can be achieved by submitting the obtained core-shell microcapsule slurry to a drying step, such as spray-drying, to provide the microcapsules as such, i.e. in a powdery form.

It is understood that any standard method known by a person skilled in the art to perform such drying is also applicable. In particular the slurry may be spray-dried preferably in the presence of a polymeric carrier material such as polyvinyl acetate, polyvinyl alcohol, dextrins, natural or modified starch, vegetable gums, pectins, xanthans, alginates, carragenans or cellulose derivatives to provide microcapsules in a powder form.

According to a particular embodiment, the carrier material contains free perfume oil which can be the same or different from the perfume from the core of the microcapsules.

### Process for Preparing Polyamide Microcapsules

Another object of the invention is a process for preparing a polyamide core-shell microcapsule slurry comprising the steps of:
a) Dispersing an oil phase comprising a hydrophobic material and at least one acyl chloride into a dispersing phase to form a two-phases dispersion;
b) Performing a curing step to form microcapsules in the form of a slurry;
wherein at least one stabilizer is added in the oil phase and/or in the dispersing phase, and
wherein at least one co-oligopeptide is added in the dispersing phase and/or in the oil phase and/or in the two-phases dispersion.

According to an embodiment, the process comprises the steps of:
a) Dispersing an oil phase comprising a hydrophobic material and at least one acyl chloride into a water phase to form an oil-in water emulsion;
b) Performing a curing step to form microcapsules in the form of a slurry;
wherein at least one stabilizer is added in the oil phase and/or in the water phase, and
wherein at least one co-oligopeptide is added in the water phase and/or in the oil phase and/or in the oil-in-water emulsion.

The embodiments disclosed previously for the polyamide core-shell microcapsules, more particularly regarding the hydrophobic material, the co-oligopeptide, the acyl chloride, also apply for the process for preparing said microcapsules.

According to an embodiment, the dispersing phase comprises, preferably consists of water.

According to an embodiment, the dispersing phase is a water phase.

According to an embodiment, the two-phases dispersion is an oil-in-water emulsion.

According to an embodiment, the dispersing phase comprises water and an alcohol such as glycerol, 1,4-butanediol, ethylene glycol and mixtures thereof.

According to an embodiment, the pH of the dispersing phase is comprised between 7 and 13, particularly between 9 and 11.

The acyl chloride can be dissolved/dispersed directly in the perfume oil or can be pre-dispersed or pre-dissolved in an inert solvent or any inert perfumery solvent/ingredient such as benzyl benzoate, triethyl citrate, ethyl acetate, hexyl salicylate or Neobee before mixing with the perfume oil.

According to the invention, a stabilizer is added in the dispersing phase and/or the oil phase to form the emulsion.

By “stabilizer”, it is meant a compound capable to stabilize oil/dispersing phase interface (typically oil/water interface) as an emulsion.

“Stabilizer” or “emulsifier” can be used indifferently in the present invention.

According to an embodiment, the stabilizer is a colloidal stabilizer.

The colloidal stabilizer can be a polymeric emulsifier (standard emulsion), a surfactant, or solid particles (Pickering emulsion).

By “polymeric emulsifier”, it meant an emulsifier having both a polar group with an affinity for the dispersing phase (typically water) (hydrophilic) and a nonpolar group with an affinity for oil (hydrophobic). The hydrophilic part will dissolve in the dispersing phase and the hydrophobic part will dissolve in the oil phase providing a film around droplets.
By “surfactant”, it meant a substance with a polar and a non-polar group that is added to the liquid to reduce the liquid surface tension.
According to an embodiment, the stabilizer is chosen in the group consisting of inorganic particles, polymeric emulsifier such as polysaccharides, proteins, glycoproteins, and mixtures thereof.

When the stabilizer is solid particles, it can be chosen in the group consisting of calcium phosphate, silica, silicates, titanium dioxide, aluminium oxide, zinc oxide, iron oxide, mica, kaolin, montmorillonite, laponite, bentonite, perlite, dolomite, diatomite, vermiculite, hectorite, gibbsite, illite, kaolinite, aluminosilicates, gypsum, bauxite, magnesite, talc, magnesium carbonate, calcium carbonate, diatomaceous earth and mixtures thereof.

According to a particular embodiment, the stabilizer is a biopolymer. By “biopolymers” it is meant biomacromolecules produced by living organisms. Biopolymers are characterized by molecular weight distributions ranging from 1,000 (1 thousand) to 1,000,000,000 (1 billion) Daltons. These macromolecules may be carbohydrates (sugar based) or proteins (amino-acid based) or a combination of both (gums) and can be linear or branched.

According to an embodiment, the polymeric emulsifier is chosen in the group consisting of gum Arabic, modified starch, polyvinyl alcohol, polyvinylpyrolidone (PVP), carboxymethylcellulose (CMC), anionic polysaccharides, acrylamide copolymer, protein such as soy protein, rice protein, whey protein, white egg albumin, sodium caseinate, gelatin, bovine serum albumin, hydrolyzed soy protein, hydrolyzed sericin, pseudocollagen, silk protein, sericin powder, potato protein, chickpea protein, pea protein, algae protein, faba bean protein, barley protein, oat protein, wheat gluten protein, lupin protein, and mixtures thereof.

Potato proteins are typically extracted from potato tuber (Solanum tuberosum). According to an embodiment, the potato protein is a native potato protein and preferably patatin.

According to an embodiment, at least one salt is added in the dispersing phase and/or in the oil phase and/or in the two-phases dispersion. The presence of a salt can enhance the stability of the polyamide shell.

A salt can be particularly added when a protein is used as a stabilizer.

The salt is preferably used in an amount comprised between 0.01% and 10% by weight based on the two-phases dispersion.

The salt added (typically in the dispersing phase) can be chosen in the group consisting of calcium, zinc, sodium, potassium, lithium, magnesium, aluminum, iron, manganese, copper, titanium, barium, sulphates, phosphates, nitrates, bromides, chlorides, iodides, acetates, and ammonium salts.

According to an embodiment, the salt is chosen in the group consisting of CaCl₂, NaCl, KCl, ZnCl₂, ZnSO₄, Zn(NO₃)₂, LiCl, Ca(NO₃)₂, MgCl₂, CaBr₂, CaI₂, NaBr, NaI, NaNO₃, KBr, KI, KNO₃, LiBr, LiI, MgBr₂, CuCl₂, FeCl₂, FeCl₃, TiCl₄, MnCl₂, and mixtures thereof.

According to an embodiment, the salt is chosen in the group consisting of CaCl₂), NaCl, KCl, ZnCl₂, LiCl, Ca(NO₃)₂, MgCl₂, and mixtures thereof.

According to a particular embodiment, the stabilizer is not a protein. According to an embodiment, no protein is added at any stage of the process.

According to an embodiment, the stabilizer is used in a concentration between 0.05% to 20%, preferably between 0.1 to 5%, by weight based on the two-phases dispersion.

According to an embodiment, the acyl chloride is used in a concentration between 0.1% to 50%, preferably between 0.5 to 15%, by weight based on the oil phase.

According to an embodiment, the pH of the stabilizer solution is comprised between 7 and 13, particularly between 9 and 11.

According to an embodiment, in addition to the co-oligopeptide, an amino compound, is added in the dispersing phase and/or in the two-phases dispersion.

The amino compound can be an amino acid and is preferably chosen in the group consisting of lysine, arginine, histidine, tryptophan, ornithine, glutamine, asparagine, citrulline, and mixtures thereof.

The amino compound can be added typically in an amount comprised between 0.1 to 10% by weight based on the two-phases dispersion.

According to another embodiment, the amino-compound can be chosen in the group consisting of 1,3-diaminopropane, 1,4-diaminobutane, 1,6-diaminohexane, 1,5-diaminopentane, 1,7-diaminoheptane, 1,8-diaminooctane, 1,10-diaminodecane, 1,9-diaminononane, 1,12-diaminododecane, 4,9-dioxa-1,12-dodecanediamine, 3,3′-diamino-N-methyldipropylamine, xylylene diamine, 1,2-diaminocyclohexane, 1,4-diaminocyclohexane, L-lysine ethyl ester, Jeffamine®, ethylene diamine, diethylene triamine, spermine, spermidine, polyamidoamine (PAMAM), guanidine carbonate, chitosan, oligo-chitosan, tris-(2-aminoethyl)amine, 3-aminopropyltriethoxysilane, arginine alkyl esters, histidine alkyl esters, tryptophan alkyl esters, ornithine alkyl esters, polylysine, polyhistidine, polyornithine, and mixtures thereof.

According to an embodiment, no additional amino compound is added at any stage of the process.

According to one embodiment, at least one further polyfunctional monomer is added to the process, wherein the at least one further polyfunctional monomer is not an acyl chloride. According to an embodiment, the at least one further polyfunctional monomer is added to the oil phase in step a) and/or to the dispersing phase in step b), preferably to the oil phase in step a).

According to an embodiment, the polyfunctional monomer is chosen in the group consisting of at least one isocyanate, anhydride or maleic anhydride, epoxide, (meth) acrylate monomers, alkoxysilane, and mixtures thereof.

Suitable polyisocyanates used according to the invention can include aromatic polyisocyanate, aliphatic polyisocyanate and mixtures thereof. Said polyisocyanate comprises at least 2, preferably at least 3 but may comprise up to 6, or even only 4, isocyanate functional groups. According to a particular embodiment, a triisocyanate (3 isocyanate functional group) is used.

According to one embodiment, said polyisocyanate is an aromatic polyisocyanate.

The term “aromatic polyisocyanate” is meant here as encompassing any polyisocyanate comprising an aromatic moiety. Preferably, it comprises a phenyl, a toluyl, a xylyl, a naphthyl or a diphenyl moiety, more preferably a toluyl or a xylyl moiety. Preferred aromatic polyisocyanates are biurets, polyisocyanurates and trimethylol propane adducts of diisocyanates, more preferably comprising one of the above-cited specific aromatic moieties. More preferably, the aromatic polyisocyanate is a polyisocyanurate of toluene diisocyanate (commercially available from Bayer under the tradename Desmodur® RC), a trimethylol propane-adduct of toluene diisocyanate (commercially available from Bayer under the tradename Desmodur® L75), a trimethylol propane-adduct of xylylene diisocyanate (commercially available from Mitsui Chemicals under the tradename Takenate® D-110N). In a most preferred embodiment, the aromatic polyisocyanate is a trimethylol propane-adduct of xylylene diisocyanate.

According to another embodiment, said polyisocyanate is an aliphatic polyisocyanate. The term “aliphatic polyisocyanate” is defined as a polyisocyanate which does not comprise any aromatic moiety. Preferred aliphatic polyisocyanates are a trimer of hexamethylene diisocyanate, a trimer of isophorone diisocyanate, a trimethylol propane-adduct of hexamethylene diisocyanate (available from Mitsui Chemicals) or a biuret of hexamethylene diisocyanate (commercially available from Bayer under the tradename Desmodur® N 100), among which a biuret of hexamethylene diisocyanate is even more preferred.

According to another embodiment, the at least one polyisocyanate is in the form of a mixture of at least one aliphatic polyisocyanate and of at least one aromatic polyisocyanate, both comprising at least two or three isocyanate functional groups, such as a mixture of a biuret of hexamethylene diisocyanate with a trimethylol propane-adduct of xylylene diisocyanate, a mixture of a biuret of hexamethylene diisocyanate with a polyisocyanurate of toluene diisocyanate and a mixture of a biuret of hexamethylene diisocyanate with a trimethylol propane-adduct of toluene diisocyanate. Most preferably, it is a mixture of a biuret of hexamethylene diisocyanate with a trimethylol propane-adduct of xylylene diisocyanate. Preferably, when used as a mixture the molar ratio between the aliphatic polyisocyanate and the aromatic polyisocyanate is ranging from 80:20 to 10:90.

According to an embodiment, the additional polyfunctional monomer is present in amounts representing from 0.1 to 15%, preferably from 0.5 to 10% and more preferably from 0.8 to 6%, and even more preferably between 1 and 3% by weight based on the total amount of the oil phase and/or of the dispersing phase.

According an embodiment, the hydrophobic material represents between about 10% and 99% by weight, relative to the total weight of the oil phase. According another embodiment, the hydrophobic material represents between about 10% and 80% by weight, relative to the total weight of the oil phase. According another embodiment, the hydrophobic material represents between about 10% and 60% by weight, relative to the total weight of the oil phase. According another embodiment, the hydrophobic material represents between about 15% and 45% by weight, relative to the total weight of the oil phase.

The curing step can be carried out typically at a temperature between 5° C. and 90° C., preferably between 40° C. and 80° C. under stirring for 5 min to 40 hours, preferably between 30 min to 24 hours, to complete the reaction and form microcapsules in the form of a slurry. However, the heating step can be omitted.

### Multiple Capsule System

According to an embodiment, the microcapsules of the invention (first type of microcapsule) can be used in combination with a second type of microcapsules.

Another object of the invention is a microcapsule delivery system comprising:
the microcapsules of the present invention as a first type of microcapsules, and
a second type of microcapsules, wherein the first type of microcapsules and the second type of microcapsules differ in their hydrophobic material and/or their wall material and/or in their coating material.

According to a particular embodiment, the microcapsule delivery system is in the form of a slurry.

The wall of the second type of microcapsules can vary. As non-limiting examples, the polymer shell of the second type of microcapsules comprises a material selected from the group consisting of polyurea, polyurethane, polyamide, polyhydroxyalkanoates, polyacrylate, polyesters, polyaminoesters, polyepoxides, organosilicon, polycarbonate, polysulfonamide, gelatin/gum arabic shell wall, and mixtures thereof.

The second type of microcapsule can comprise an oil-based core comprising a hydrophobic active, preferably perfume, and a composite shell comprising a first material and a second material, wherein the first material and the second material are different, the first material is a coacervate, the second material is a polymeric material. In a particular embodiment, the weight ratio between the first material and the second material is comprised between 50:50 and 99.9:0.1. In a particular embodiment, the coacervate comprises a first polyelectrolyte, preferably selected among proteins (such as gelatin), polypeptides or polysaccharides (such as chitosan), most preferably Gelatin and a second polyelectrolyte, preferably alginate salts, cellulose derivatives guar gum, pectinate salts, carrageenan, polyacrylic and methacrylic acid or xanthan gum, or yet plant gums such as acacia gum (Gum Arabic), most preferably Gum Arabic. The coacervate first material can be hardened chemically using a suitable cross-linker such as glutaraldehyde, glyoxal, formaldehyde, tannic acid or genipin or can be hardenedenzymatically using an enzyme such as transglutaminase. The second polymeric material can be selected from the group consisting of polyurea, polyurethane, polyamide, polyester, polyacrylate, organosilicon, polycarbonate, polysulfonamide, and mixtures thereof, preferably polyurea and/or polyurethane. The second material is preferably present in an amount less than 3 wt. %, preferably less than 1 wt. % based on the total weight of the second type of microcapsule slurry.

As non-limiting examples, the shell of the second type of microcapsules can be polyurea-based or polyurethane-based. The shell of the second type of microcapsules can also be hybrid, namely organic-inorganic such as a hybrid shell composed of at least two types of inorganic particles that are cross-linked, or yet a shell resulting from the hydrolysis and condensation reaction of a polyalkoxysilane macro-monomeric composition.

According to another aspect the shell of the second type of microcapsules is polyurea-based made from, for example but not limited to isocyanate-based monomers and amine-containing crosslinkers such as guanidine carbonate and/or guanazole. Certain polyurea microcapsules comprise a polyurea wall which is the reaction product of the polymerisation between at least one polyisocyanate comprising at least two isocyanate functional groups and at least one reactant selected from the group consisting of an amine (for example a water-soluble guanidine salt and guanidine); a colloidal stabilizer or emulsifier; and an encapsulated perfume. However, the use of an amine can be omitted. According to a particular aspect, the colloidal stabilizer includes an aqueous solution of between 0.1% and 0.4% of polyvinyl alcohol, between 0.6% and 1% of a cationic copolymer of vinylpyrrolidone and of a quaternized vinylimidazol (all percentages being defined by weight relative to the total weight of the colloidal stabilizer). According to another aspect, the emulsifier is an anionic or amphiphilic biopolymer, which may be, in one aspect, chosen from the group consisting of gum Arabic, soy protein, gelatin, sodium caseinate and mixtures thereof.

According to a particular embodiment, the second type of microcapsules comprises:
an oil-based core comprising a hydrophobic active, preferably perfume, and
a polyamide shell comprising or being obtainable from:
   an acyl chloride, preferably in an amount comprised between 5 and 98%, preferably between 20 and 98%, more preferably between 30 and 85% w/w
   a first amino compound, preferably in an amount comprised between 1% and 50% w/w, preferably between 7 and 40% w/w;
   a second amino compound, preferably in an amount comprised between 1% and 50% w/w, preferably between 2 and 25% w/w
   optionally, a stabilizer, preferably a biopolymer, preferably in an amount comprised between 0 and 90%, preferably between 0.1 and 75%, more preferably between 1 and 70%,
   optionally, a carbohydrate.

According to a particular embodiment, the second type of microcapsules comprises:
an oil-based core comprising a hydrophobic active, preferably perfume, and
a polyamide shell comprising or being obtainable from:
   an acyl chloride,
   a first amino-compound being an amino-acid, preferably chosen in the group consisting of L-Lysine, L-Arginine, L-Histidine, L-Tryptophane and/or mixtures thereof.
   a second amino-compound, preferably chosen in the group consisting of ethylene diamine, diethylene triamine, cystamine and/or mixtures thereof, and
   a biopolymer, preferably chosen in the group consisting of potato protein, chickpea protein, pea protein, algae protein, faba bean protein, barley protein, oat protein, wheat gluten protein, lupin protein, soy protein, rice protein, whey protein, white egg albumin, casein, sodium caseinate, gelatin (preferably fish gelatin), bovine serum albumin, hydrolyzed soy protein, hydrolyzed sericin, pseudocollagen, silk protein, sericin powder, gelatin and mixtures thereof,
   optionally a carbohydrate, preferably selected from the group consisting of anionic salt of alginic acid, preferably alginic acid sodium salt, pectin, lignin, anionic modified starch, carboxymethylcellulose, carrageenan and mixtures thereof.

According to another aspect, the shell of the second type of microcapsules is polyurea- or polyurethane-based. Examples of processes for the preparation of polyurea and polyurethane-based microcapsule slurry are for instance described in International Patent Application Publication No. WO2007/004166, European Patent Application Publication No. EP 2300146, and European Patent Application Publication No. EP25799. Typically a process for the preparation of polyurea or polyurethane-based microcapsule slurry include the following steps:
a) Dissolving at least one polyisocyanate having at least two isocyanate groups in an oil to form an oil phase;
b) Preparing an aqueous solution of an emulsifier or colloidal stabilizer to form a dispersing phase;
c) Adding the oil phase to the dispersing phase to form an oil-in-water dispersion, wherein the mean droplet size is comprised between 1 and 500 μm, preferably between 5 and 50 μm; and
d) Applying conditions sufficient to induce interfacial polymerisation and form microcapsules in form of a slurry.

### Perfuming Composition and Consumer Products

The microcapsules of the invention can be used in combination with active ingredients. An object of the invention is therefore a composition comprising:
(i) microcapsules or microcapsule slurry as defined above;
(ii) an active ingredient, preferably chosen in the group consisting of a cosmetic ingredient, skin caring ingredient, perfume ingredient, flavor ingredient, malodour counteracting ingredient, bactericide ingredient, fungicide ingredient, pharmaceutical or agrochemical ingredient, a sanitizing ingredient, an insect repellent or attractant, and mixtures thereof.

The microcapsules of the invention show a good performance in terms of stability in challenging medium.

Another object of the present invention is a perfuming composition comprising:
(i) microcapsules or microcapsule slurry as defined above, wherein the oil comprises a perfume;
(ii) at least one ingredient selected from the group consisting of a perfumery carrier, a perfumery co-ingredient and mixtures thereof;
(iii) optionally at least one perfumery adjuvant.
   As liquid perfumery carrier one may cite, as non-limiting examples, an emulsifying system, i.e. a solvent and a surfactant system, or a solvent commonly used in perfumery. A detailed description of the nature and type of solvents commonly used in perfumery cannot be exhaustive. However, one can cite as non-limiting examples solvents such as dipropyleneglycol, diethyl phthalate, isopropyl myristate, benzyl benzoate, 2-(2-ethoxyethoxy)-1-ethanol or ethyl citrate, which are the most commonly used. For the compositions which comprise both a perfumery carrier and a perfumery co-ingredient, other suitable perfumery carriers than those previously specified, can be also ethanol, water/ethanol mixtures, limonene or other terpenes, isoparaffins such as those known under the trademark Isopar® (origin: Exxon Chemical) or glycol ethers and glycol ether esters such as those known under the trademark Dowanol® (origin: Dow Chemical Company). By “perfumery co-ingredient”it is meant here a compound, which is used in a perfuming preparation or a composition to impart a hedonic effect and which is not a microcapsule as defined above. In other words such a co-ingredient, to be considered as being a perfuming one, must be recognized by a person skilled in the art as being able to at least impart or modify in a positive or pleasant way the odor of a composition, and not just as having an odor.
   The nature and type of the perfuming co-ingredients present in the perfuming composition do not warrant a more detailed description here, which in any case would not be exhaustive, the skilled person being able to select them on the basis of his general knowledge and according to the intended use or application and the desired organoleptic effect. In general terms, these perfuming co-ingredients belong to chemical classes as varied as alcohols, lactones, aldehydes, ketones, esters, ethers, acetates, nitriles, terpenoids, nitrogenous or sulphurous heterocyclic compounds and essential oils, and said perfuming co-ingredients can be of natural or synthetic origin. Many of these co-ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of perfumery. It is also understood that said co-ingredients may also be compounds known to release in a controlled manner various types of perfuming compounds also known as properfume or profragrance. Non-limiting examples of suitable properfumes may include 4-(dodecylthio)-4-(2,6,6-trimethyl-2-cyclohexen-1-yl)-2-butanone, 4-(dodecylthio)-4-(2,6,6-trimethyl-1-cyclohexen-1-yl)-2-butanone, trans-3-(dodecylthio)-1-(2,6,6-trimethyl-3-cyclohexen-1-yl)-1-butanone, 2-(dodecylthio)octan-4-one, 2-phenylethyl oxo(phenyl)acetate, 3,7-dimethylocta-2,6-dien-1-yl oxo(phenyl)acetate, (Z)-hex-3-en-1-yl oxo(phenyl)acetate, 3,7-dimethyl-2,6-octadien-1-yl hexadecanoate, bis(3,7-dimethylocta-2,6-dien-1-yl) succinate, (2-((2-methylundec-1-en-1-yl)oxy)ethyl)benzene, 1-methoxy-4-(3-methyl-4-phenethoxybut-3-en-1-yl)benzene, (3-methyl-4-phenethoxybut-3-en-1-yl)benzene, 1-(((Z)-hex-3-en-1-yl)oxy)-2-methylundec-1-ene, (2-((2-methylundec-1-en-1-yl)oxy)ethoxy)benzene, 2-methyl-1-(octan-3-yloxy)undec-1-ene, 1-methoxy-4-(1-phenethoxyprop-1-en-2-yl)benzene, 1-methyl-4-(1-phenethoxyprop-1-en-2-yl)benzene, 2-(1-phenethoxyprop-1-en-2-yl)naphthalene, (2-phenethoxyvinyl)benzene, 2-(1-((3,7-dimethyloct-6-en-1-yl)oxy)prop-1-en-2-yl)naphthalene, (2-((2-pentylcyclopentylidene)methoxy)ethyl)benzene, 4-allyl-2-methoxy-1-((2-methoxy-2-phenylvinyl)oxy)benzene, (2-((2-heptylcyclopentylidene)methoxy)ethyl)benzene, 1-isopropyl-4-methyl-2-((2-pentylcyclopentylidene)methoxy)benzene, 2-methoxy-1-((2-pentylcyclopentylidene)methoxy)-4-propylbenzene, 3-methoxy-4-((2-methoxy-2-phenylvinyl)oxy)benzaldehyde, 4-((2-(hexyloxy)-2-phenylvinyl)oxy)-3-methoxybenzaldehyde or a mixture thereof.

By “perfumery adjuvant” we mean here an ingredient capable of imparting additional added benefit such as a color, a particular light resistance, chemical stability, etc. A detailed description of the nature and type of adjuvant commonly used in perfuming bases cannot be exhaustive, but it has to be mentioned that said ingredients are well known to a person skilled in the art.

Preferably, the perfuming composition according to the invention comprises between 0.01 and 30% by weight of microcapsules as defined above.

The invention's microcapsules can advantageously be used in many application fields and used in consumer products. Microcapsules can be used in liquid form applicable to liquid consumer products as well as in powder form, applicable to powder consumer products.

According to a particular embodiment, the consumer product as defined above is liquid and comprises:
a) from 2 to 65% by weight, relative to the total weight of the consumer product, of at least one surfactant;
b) water or a water-miscible hydrophilic organic solvent; and
c) a microcapsule slurry or microcapsules as defined above,
d) optionally non-encapsulated perfume.

According to a particular embodiment, the consumer product as defined above is in a powder form and comprises:
a) from 2 to 65% by weight, relative to the total weight of the consumer product, of at least one surfactant;
b) a microcapsule powder as defined above.
c) optionally perfume powder that is different from the microcapsules defined above.

In the case of microcapsules including a perfume oil-based core, the products of the invention, can in particular be of used in perfumed consumer products such as product belonging to fine fragrance or “functional” perfumery. Functional perfumery includes in particular personal-care products including hair-care, body cleansing, skin care, hygiene-care as well as home-care products including laundry care, surface care and air care.

Consequently, another object of the present invention consists of a perfumed consumer product comprising as a perfuming ingredient, the microcapsules defined above or a perfuming composition as defined above. The perfume element of said consumer product can be a combination of perfume microcapsules as defined above and free or non-encapsulated perfume, as well as other types of perfume microcapsules than those here-disclosed.

In particular a liquid consumer product comprising:
a) from 2 to 65% by weight, relative to the total weight of the consumer product, of at least one surfactant;
b) water or a water-miscible hydrophilic organic solvent; and
c) a perfuming composition as defined above is another object of the invention.

Also a powder consumer product comprising
(a) from 2 to 65% by weight, relative to the total weight of the consumer product, of at least one surfactant; and
(b) a perfuming composition as defined above is part of the invention.

The invention's microcapsules can therefore be added as such or as part of an invention's perfuming composition in a perfumed consumer product.

For the sake of clarity, it has to be mentioned that, by “perfumed consumer product” it is meant a consumer product which is expected to deliver among different benefits a perfuming effect to the surface to which it is applied (e.g. skin, hair, textile, paper, or home surface) or in the air (air-freshener, deodorizer etc.). In other words, a perfumed consumer product according to the invention is a manufactured product which comprises a functional formulation also referred to as “base”, together with benefit agents, among which an effective amount of microcapsules according to the invention.

The nature and type of the other constituents of the perfumed consumer product do not warrant a more detailed description here, which in any case would not be exhaustive, the skilled person being able to select them on the basis of his general knowledge and according to the nature and the desired effect of said product. Base formulations of consumer products in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

Non-limiting examples of suitable perfumed consumer products can be a perfume, such as a fine perfume, a cologne, an after-shave lotion, a body-splash; a fabric care product, such as a liquid or solid detergent, tablets and unit dose (single or multi chambers), a fabric softener, a dryer sheet, a fabric refresher, an ironing water, or a bleach; a personal-care product, such as a hair-care product (e.g. a shampoo, hair conditioner, a coloring preparation or a hair spray), a cosmetic preparation (e.g. a vanishing cream, body lotion or a deodorant or antiperspirant), or a skin-care product (e.g. a perfumed soap, shower or bath mousse, body wash, oil or gel, bath salts, or a hygiene product); an air care product, such as an air freshener or a “ready to use” powdered air freshener; or a home care product, such all-purpose cleaners, liquid or power or tablet dishwashing products, toilet cleaners or products for cleaning various surfaces, for example sprays & wipes intended for the treatment/refreshment of textiles or hard surfaces (floors, tiles, stone-floors etc.); a hygiene product such as sanitary napkins, diapers, toilet paper.

Another object of the invention is a consumer product comprising:
a personal care active base, and
microcapsules or microcapsule slurry as defined above or the perfuming composition as defined above,
wherein the consumer product is in the form of a personal care composition.

Personal care active bases in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

The personal care composition is preferably chosen in the group consisting of a hair-care product (e.g. a shampoo, hair conditioner, a coloring preparation or a hair spray), a cosmetic preparation (e.g. a vanishing cream, body lotion or a deodorant or antiperspirant), or a skin-care product (e.g. a perfumed soap, shower or bath mousse, body wash, oil or gel, bath salts, or a hygiene product);

Another object of the invention is a consumer product comprising:
a home care or a fabric care active base, and
microcapsules or microcapsule slurry as defined above or the perfuming composition as defined above,
   wherein the consumer product is in the form of a home care or a fabric care composition.

Home care or fabric care active bases in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

Preferably, the consumer product comprises from 0.1 to 15 wt %, more preferably between 0.2 and 5 wt % of the microcapsules of the present invention, these percentages being defined by weight relative to the total weight of the consumer product. Of course, the above concentrations may be adapted according to the benefit effect desired in each product.

For liquid consumer product mentioned below, by “active base”, it should be understood that the active base includes active materials (typically including surfactants) and water.

For solid consumer product mention below, by “active base”, it should be understood that the active base includes active materials (typically including surfactants) and auxiliary agents (such as bleaching agents, buffering agent; builders; soil release or soil suspension polymers; granulated enzyme particles, corrosion inhibitors, antifoaming, sud suppressing agents; dyes, fillers, and mixtures thereof).

### Fabric Softener

An object of the invention is a consumer product in the form of a fabric softener composition comprising:
a fabric softener active base; preferably comprising at least one active material chosen in the group consisting of dialkyl quaternary ammonium salts, dialkyl ester quaternary ammonium salts (esterquats), Hamburg esterquat (HEQ), TEAQ (triethanolamine quat), silicones and mixtures thereof, the active base being used preferably in an amount comprised between 85 and 99.95% by weight based on the total weight of the composition,
microcapsules or a microcapsule slurry as defined above, preferably in an amount comprised between 0.05 to 15 wt %, more preferably between 0.1 and 5 wt % by weight based on the total weight of the composition,
optionally free perfume oil.

### Liquid Detergent

An object of the invention is a consumer product in the form of a liquid detergent composition comprising:
a liquid detergent active base; preferably comprising at least one active material chosen in the group consisting of anionic surfactant such as alkylbenzenesulfonate (ABS), secondary alkyl sulfonate (SAS), primary alcohol sulfate (PAS), lauryl ether sulfate (LES), methyl ester sulfonate (MES) and nonionic surfactant such as alkyl amines, alkanolamide, fatty alcohol poly(ethylene glycol) ether, fatty alcohol ethoxylate (FAE), ethylene oxide (EO) and propylene oxide (PO) copolymers, amine oxydes, alkyl polyglucosides, alkyl polyglucosamides, the active base being used preferably in an amount comprised between 85 and 99.95% by weight based on the total weight of the composition,
microcapsules or a microcapsule slurry as defined above, preferably in an amount comprised between 0.05 to 15 wt %, more preferably between 0.1 and 5 wt % by weight based on the total weight of the composition,
optionally free perfume oil.

### Solid Detergent

An object of the invention is a consumer product in the form of a solid detergent composition comprising:
a solid detergent active base; preferably comprising at least one active material chosen in the group consisting of anionic surfactant such as alkylbenzenesulfonate (ABS), secondary alkyl sulfonate (SAS), primary alcohol sulfate (PAS), lauryl ether sulfate (LES), methyl ester sulfonate (MES) and nonionic surfactant such as alkyl amines, alkanolamide, fatty alcohol poly(ethylene glycol) ether, fatty alcohol ethoxylate (FAE), ethylene oxide (EO) and propylene oxide (PO) copolymers, amine oxydes, alkyl polyglucosides, alkyl polyglucosamides, the active base being used preferably in an amount comprised between 85 and 99.95% by weight based on the total weight of the composition,
a microcapsule powder or microcapsule slurry as defined above, preferably in an amount comprised between 0.05 to 15 wt %, more preferably between 0.1 and 5 wt % by weight based on the total weight of the composition,
optionally free perfume oil.

### Shampoo/Shower Gel

An object of the invention is a consumer product in the form of a shampoo or a shower gel composition comprising:
a shampoo or a shower gel active base; preferably comprising at least one active material chosen in the group consisting of sodium alkylether sulfate, ammonium alkylether sulfates, alkylamphoacetate, cocamidopropyl betaine, cocamide MEA, alkylglucosides and aminoacid based surfactants and mixtures thereof, the active base being used preferably in an amount comprised between 85 and 99.95% by weight based on the total weight of the composition,
microcapsules or a microcapsule slurry as defined above, preferably in an amount comprised between 0.05 to 15 wt %, more preferably between 0.1 and 5 wt % by weight based on the total weight of the composition,
optionally free perfume oil.

### Rinse-Off Conditioner

An object of the invention is a consumer product in the form of a rinse-off conditioner composition comprising:
a rinse-off conditioner active base; preferably comprising at least one active material chosen in the group consisting of cetyltrimonium chloride, stearyl trimonium chloride, benzalkonium chloride, behentrimonium chloride and mixture thereof, the active base being used preferably in an amount comprised between 85 and 99.95% by weight based on the total weight of the composition,
microcapsules or a microcapsule slurry as defined above, preferably in an amount comprised between 0.05 to 15 wt %, more preferably between 0.1 and 5 wt % by weight based on the total weight of the composition,
optionally free perfume oil.

### Solid Scent Booster

An object of the invention is a consumer product in the form of a solid scent booster composition comprising:
a solid carrier, preferably chosen in the group consisting of urea, sodium chloride, sodium sulphate, sodium acetate, zeolite, sodium carbonate, sodium bicarbonate, clay, talc, calcium carbonate, magnesium sulfate, gypsum, calcium sulfate, magnesium oxide, zinc oxide, titanium dioxide, calcium chloride, potassium chloride, magnesium chloride, zinc chloride, saccharides such as sucrose, mono-, di-, and polysaccharides and derivatives such as starch, cellulose, methyl cellulose, ethyl cellulose, propyl cellulose, polyols/sugar alcohols such as sorbitol, maltitol, xylitol, erythritol, and isomalt, PEG, PVP, citric acid or any water soluble solid acid, fatty alcohols or fatty acids and mixtures thereof,
microcapsules or a microcapsule slurry as defined above, in a powdered form, preferably in an amount comprised between 0.05 to 15 wt %, more preferably between 0.1 and 5 wt % by weight based on the total weight of the composition,
optionally free perfume oil.

### Liquid Scent Booster

An object of the invention is a consumer product in the form of a liquid scent booster composition comprising:
an aqueous phase,
a surfactant system essentially consisting of one or more than one non-ionic surfactant, wherein the surfactant system has a mean HLB between 10 and 14, preferably chosen in the group consisting of ethoxylated aliphatic alcohols, POE/PPG (polyoxyethylene and polyoxypropylene) ethers, mono and polyglyceryl esters, sucrose ester compounds, polyoxyethylene hydroxylesters, alkyl polyglucosides, amine oxides and combinations thereof;
a linker chosen in the group consisting of alcohols, salts and esters of carboxylic acids, salts and esters of hydroxyl carboxylic acids, fatty acids, fatty acid salts, glycerol fatty acids, surfactant having an HLB less than 10 and mixtures thereof, and
microcapsules or a microcapsule slurry as defined above, in the form of a slurry, preferably in an amount comprised between 0.05 to 15 wt %, more preferably between 0.1 and 5 wt % by weight based on the total weight of the composition,
optionally free perfume oil.

### Hair Coloration

An object of the invention is a consumer product in the form of an oxidative hair coloring composition comprising:
an oxidizing phase comprising an oxidizing agent and an alkaline phase comprising an alkakine agent, a dye precursor and a coupling compound; wherein said dye precursor and said coupling compound form an oxidative hair dye in the presence of the oxidizing agent, preferably in an amount comprised between 85 and 99.95% by weight based on the total weight of the composition,
microcapsules or a microcapsule slurry as defined above, preferably in an amount comprised between 0.05 to 15 wt %, more preferably between 0.1 and 5 wt % by weight based on the total weight of the composition,
optionally free perfume oil

### Perfuming Composition

According to a particular embodiment, the consumer product is in the form of a perfuming composition comprising:
0.1 to 30%, preferably 0.1 to 20% of microcapsules or a microcapsule slurry as defined previously,
0 to 40%, preferably 3-40% of perfume, and
20-90%, preferably 40-90% of ethanol, by weight based on the total weight of the perfuming composition.

The invention will now be further described by way of examples. It will be appreciated that the invention as claimed is not intended to be limited in any way by these examples.

### EXAMPLES

### Example 1

### Co-Oligopeptide 1 Mixture Preparation

62 mL 0.73 M lysine ethyl ester dihydrochloride (Lys) and leucine ethyl ester hydrochloride (Leu) (molar ratio of Lys:Leu=4:1) was prepared as the reaction solution and heated to 40° C. 4 mL bromelain aqueous solution (initial enzyme activity=4.5×10⁵ CDU/mL) (Origin: Enzyme Development Corporation) was added to the reaction solution. The reaction solution was stirred at 40° C. for 1.5 h (pH=7.8), then heated to 80° C. for 15 min, cooled down to room temperature, centrifuged or filtered, and lyophilized or vacuum dried. The resultant solids, called co-oligopeptide 1 mixture, were used without further purification.

### Example 2

### Co-Oligopeptide 2 Mixture Preparation

48 mL 0.76 M lysine ethyl ester dihydrochloride was prepared as the reaction solution and heated to 40° C. 3 mL bromelain aqueous solution (initial enzyme activity=5.04×10⁵ CDU/mL) (Origin: Enzyme Development Corporation) was added to the reaction solution solution, which was then stirred at 40° C. for 1 h (pH=7.8). 14 mL 0.65 M leucine ethyl ester hydrochloride solution was added to the reaction solution. 1 mL bromelain aqueous solution (2.9×10⁵ CDU/mL) was added to the reaction solution, which was stirred at 40° C. for another 1 h (pH=7.8), then heated to 80° C. for 15 min, cooled down to room temperature, centrifuged or filtered, and lyophilized or vacuum dried. The resultant solids, called co-oligopeptide 2 mixture, were used without further purification.

### Example 3

### Co-Oligopeptide 3 Mixture Preparation

47 mL 0.77 M lysine ethyl ester dihydrochloride was prepared as the reaction solution and heated to 40° C. 3 mL bromelain aqueous solution (initial enzyme activity=5.04×10⁵ CDU/mL) (Origin: Enzyme Development Corporation) was added to the reaction solution, which was then stirred at 40° C. for 1 h (pH=7.8). 14 mL 0.65 M tyrosine ethyl ester hydrochloride solution was added to the reaction solution. 1 mL bromelain aqueous solution (3.8×10⁵ CDU/mL) was added to the reaction solution, which was stirred at 40° C. for another 1 h (pH=7.8), then heated to 80° C. for 15 min, cooled down to room temperature, centrifuged or filtered, and lyophilized or vacuum dried. The resultant solids, called co-oligopeptide 3 mixture, were used without further purification.

### Example 4

### Preparation of Microcapsules According to the Invention

### Microcapsules A

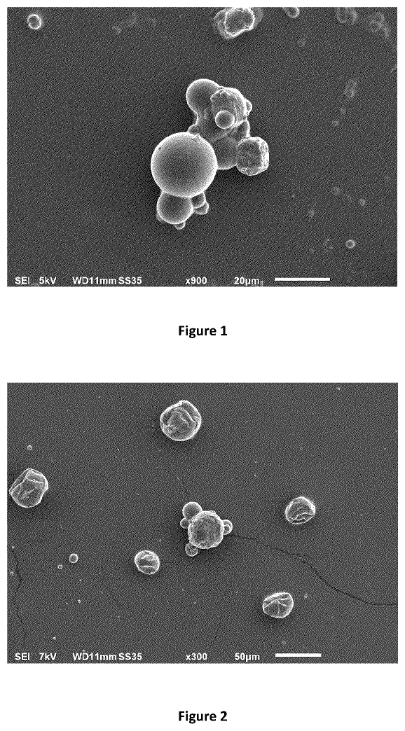

1.78 g of co-oligopeptide mixture as prepared in example 1 was dissolved in 60.8 g of water with 0.8 g gum Arabic (Superstab AA—Origin: Nexira) to form the emulsifier solution. The pH of the emulsifier solution was adjusted to 10 with 10% NaOH. 16 g of perfume oil A (see Table 1) containing 0.8 g TPCI (terephthaloyl chloride Origin: Alfa Aesar) was mixed with the emulsifier solution and homogenized at 18000 rpm for 2 min using Ultraturrax T-25. The formed oil-in-water emulsion was then stirred at 60° C. for 2 h to form a microcapsule slurry. The morphology of the capsule slurry has been observed under SEM microscope (see FIG. 1) and shows that microcapsules are formed.

TABLE 1
Perfume oil A composition

| Ingredients | % in oil |
|---|---|
| Ethyl 2-methyl-pentanoate | 3.2% |
| Eucalyptol | 7.8% |
| 2,4-Dimethyl-3-cyclohexene-1-carbaldehyde | 0.75% |
| Aldehyde C10 | 0.75% |
| Citronellyl Nitrile | 4.3% |
| Isobornyl acetate | 3% |
| 2-tert-butyl-1-cyclohexyl acetate | 9.8% |
| Citronellyl Acetate | 1.3% |
| 2-Methylundecanal | 3% |
| Diphenyloxide | 0.8% |
| Aldehyde C12 | 1.3% |
| Dicyclopentadiene acetate | 9.85% |
| Ionone beta | 3.3% |
| Undecalactone gamma | 18.75% |
| Hexyl Salicylate | 15.9% |
| Benzyl Salicylate | 16.2% |

### Microcapsules B

2.65 g of co-oligopeptide mixture as prepared in example 1 was dissolved in 60.8 g of water with 0.8 g gum arabic. The pH of the emulsifier solution was adjusted to 10 with 10% NaOH. 16 g of perfume oil A (see Table 1) containing 0.8 g TPCI was mixed with the emulsifier solution and homogenized at 18000 rpm for 2 min using Ultraturrax T-25. The formed oil-in-water emulsion was then then stirred at 60° C. for 2 h to form a microcapsule slurry.

### Microcapsules C

1.67 g co-oligopeptide mixture as prepared in example 2 was dissolved in 60 g of water with 0.8 g gum arabic to form the emulsifier solution. The pH of the emulsifier solution was adjusted to 10 with 10% NaOH. 16 g of perfume oil A (see Table 1) with 0.8 g TPCI was mixed with the emulsifier solution and homogenized at 18000 rpm for 2 min using Ultraturrax T-25. The formed oil-in-water emulsion was then stirred at 60° C. for 3 h to form a microcapsule slurry.

### Microcapsules D

2.83 g co-oligopeptide mixture as prepared in example 2 was dissolved in 54.82 g of water with 0.9 g gum arabic and 1.72 g L-lysine to form the emulsifier solution. 0.25 g CaCl₂ 2H₂O was dissolved in 1.13 g water to form the salt solution, which was then added to the emulsifier solution. 27 g of perfume oil A (see Table 1) with 1.35 g TPCI was mixed with the emulsifier solution and homogenized at 18000 rpm for 30 seconds using Ultraturrax T-25. The formed oil-in-water emulsion was then stirred at 60° C. for 2 h to form a microcapsule slurry (see FIG. 2).

### Microcapsules E

1.67 of co-oligopeptide mixture as prepared in example 3 was dissolved in 60 g of water with 0.8 g gum arabic to form the emulsifier solution. The pH of the emulsifier solution was adjusted to 10 with 10% NaOH. 16 g of perfume oil A (see Table 1) with 0.8 g TPCI was mixed with the emulsifier solution and homogenized at 18000 rpm for 2 min using Ultraturrax T-25. The formed oil-in-water emulsion was then stirred at 60° C. for 3 h to form a microcapsule slurry.

### Example 5

### Liquid Fabric Softener

A sufficient amount of microcapsule slurry A-E of the present invention is dispersed in base described below to obtain a concentration of encapsulated perfume oil at 0.22%.

TABLE 2
Liquid fabric softener composition

| Ingredients | Wt % |
|---|---|
| Stepantex VL 90A | 8.88 |
| Calcium Chloride Sol. 10% | 0.36 |
| Proxel GXL | 0.04 |
| Perfume | 1.00 |
| Water | 89.72 |
| TOTAL | 100 |

### Example 6

### Liquid Detergent Composition

A sufficient amount of microcapsule slurry A-E of the present invention is dispersed in liquid detergent base described below to obtain a concentration of encapsulated perfume oil at 0.22%.

TABLE 3
Composition of the liquid detergent formulation

| Ingredients | Concentration [wt %] |
|---|---|
| Sodium C14-17 Alkyl Sec Sulfonate¹⁾ | 7 |
| Fatty acids, C12-18 and C18-unsaturated²⁾ | 7.5 |
| C12/14 fatty alcohol polyglycol ether with 7 | 17 |
| mol EO³⁾ | |
| Triethanolamine | 7.5 |
| Propylene Glycol | 11 |
| Citric acid | 6.5 |
| Potassium Hydroxyde | 9.5 |
| Protease | 0.2 |
| Amylase | 0.2 |
| Mannanase | 0.2 |
| Acrylates/Steareth-20 Methacrylate | 6 |
| structuring Crosspolymer⁴⁾ | |
| Deionized Water | 27.4 |

¹⁾Hostapur SAS 60; Origin: Clariant
²⁾Edenor K 12-18; Origin: Cognis
³⁾Genapol LA 070; Origin: Clariant
⁴⁾Aculyn 88; Origin: Dow Chemical

### Example 7

### Rinse-Off Conditioner

A sufficient amount of microcapsule slurry A-E of the present invention is incorporated at the required dosage (corresponding to an encapsulated perfume oil at 0.5%) in the rinse-off base (see below).

TABLE 4
rinse-off conditioner composition

| | | Concentration |
|---|---|---|
| | Ingredients | [wt %] |
| A | Water deionized | 81.8 |
| | Behentrimonium Chloride ¹⁾ | 2.5 |
| | Hydroxyethylcellulose ²⁾ | 1.5 |
| B | Cetearyl Alcohol ³⁾ | 4 |
| | Glyceryl Stearate (and) PEG-100 Stearate ⁴⁾ | 2 |
| | Behentrimonium Methosulfate (and) Cetyl alcohol | 4 |
| | (and) Butylene Glycol ⁵⁾ | |
| | Ethoxy (20) Stearyl Alcohol ⁶⁾ | 1 |
| C | Amodimethicone (and) Trideceth-12 (and) | 3 |
| | Cetrimonium Chloride ⁷⁾ | |
| | Chlorhexidine Digluconate ⁸⁾ 20% aqueous solution | 0.2 |
| D | Citric acid 10% aqueous sol. till pH 3.5-4 | q.s. |
| | TOTAL: | 100 |

¹⁾ Genamin KDM P, Clariant
²⁾ Tylose H10 Y G4, Shin Etsu
³⁾ Lanette O, BASF
⁴⁾ Arlacel 165-FP-MBAL-PA-(RB), Croda
⁵⁾ Incroquat Behenyl TMS-50-MBAL-PA-(MH) HA4112, Croda
⁶⁾ SP Brij S20 MBAL-PA(RB), Croda
⁷⁾ Xiameter DC MEM-0949 Emulsion, Dow Corning
⁸⁾ Alfa Aesar

Ingredients of Phase A are mixed until a uniform mixture was obtained. Tylose is allowed to completely dissolve. Then the mixture is heated up to 70-75° C. Ingredients of Phase B are combined and melted at 70-75° C. Then ingredients of Phase B are added to Phase A with good agitation and the mixing is continued until cooled down to 60° C. Then, ingredients of Phase C are added while agitating and keeping mixing until the mixture cooled down to 40° C. The pH is adjusted with citric acid solution till pH: 3.5-4.0.

### Example 8

### Spray-Dried Microcapsules Preparation

Emulsions 1-5 having the following ingredients are prepared.

TABLE 5
Composition of Emulsions 1-5 and composition
of granulated powder 1-5 after spray-drying

| Ingredients | Emulsion 1 | Emulsion 2 | Emulsion 3 | Emulsion 4 | Emulsion 5 |
|---|---|---|---|---|---|
| Modified starch ¹⁾ | 2.6% | 2.6% | 2.6% | 12.5% | 2% |
| Maltodextrin ²⁾ | 26.8% | 22.8% | 19.3% | 0% | 19.1% |
| Maltose ³⁾ | 0% | 0% | 0% | 7.9% | 0% |
| Citric Acid | 0% | 0% | 0% | 1% | 0% |
| Tripotassium | 0% | 0% | 0% | 1.9% | 0% |
| Citrate | | | | | |
| Microcapsules | 12.0% | 24% | 37.0% | 8.9% | 56.2% |
| A-E | | | | | |
| Silica ⁴⁾ | 1.1% | 1.1% | 1.1% | 0% | 0% |
| Free Perfume | 0% | 0% | 0% | 11% | 0% |
| Water | 57.6% | 49.6% | 40.1% | 56.9% | 22.7% |
| | Granule 1 | Granule 2 | Granule 3 | Granule 4 | Granule 5 |
| Modified starch ¹⁾ | 7.5% | 7.4% | 7.2% | 31.6% | 4.9% |
| Maltodextrin ²⁾ | 77.4% | 65.5% | 53.8% | 0% | 44.7% |
| Maltose | 0% | 0% | 0% | 20.9% | |
| Citric Acid | 0% | 0% | 0% | 2.6% | 0% |
| Tripotassium | 0% | 0% | 0% | 4.9% | 0% |
| citrate | | | | | |
| Encapsulated | 0% | 0% | 0% | 28.1% | 0% |
| perfume | | | | | |
| Microcapsules A- | 12.% | 24.1% | 36.1% | 9.8% | 48.4% |
| E | | | | | |
| Silica | 3.0 | 3.0% | 2.9% | 2.0% | 2% |
| Fragrance loading | 10.1% | 20.1% | 30% | 35.8% | 40.2% |
| in powder after | | | | | |
| spray-drying | | | | | |

¹⁾ CapsulTM, Ingredion
²⁾ Maltodextrin 10DE origin: Roquette
³⁾ Maltose, Lehmann & Voss
⁴⁾ Silica, Evonik

Components for the polymeric matrix (Maltodextrin and Capsul™, or Capsul™, citric acid and tripotassium citrate) are added in water at 45-50° C. until complete dissolution.

For emulsion 4, free perfume C is added to the aqueous phase.

Microcapsules slurry is added to the obtained mixture. Then, the resulting mixture is then mixed gently at 25° C. (room temperature).

Granulated powder 1-5 are prepared by spray-drying Emulsion A-E using a Sodeva Spray Dryer (Origin France), with an air inlet temperature set to 215° C. and a throughput set to 500 ml per hour. The air outlet temperature is of 105° C. The emulsion before atomization is at ambient temperature.

### Example 9

### Liquid Scent Booster Composition

A sufficient amount of microcapsule slurry A-E is weighed and mixed in a liquid scent booster to add the equivalent of 0.2% perfume.

TABLE 6
Liquid scent booster composition

| | Amount (% wt) | | | | | |
|---|---|---|---|---|---|---|
| Ingredients | 1 | 2 | 3 | 4 | 5 | 6 |
| Water | 71.20% | 89.5% | 78.8% | 79.4% | 70% | 70% |
| Propylene | 20.30% | — | — | — | 20% | 20% |
| glycol | | | | | | |
| Polyethylene | 4.00% | 6% | | | | |
| glycol | | | | | | |
| ethers of decyl | | | | | | |
| alcohol¹⁾ | | | | | | |
| Polyethylene | | | | | 4.00% | 4.00% |
| glycol ether | | | | | | |
| of Lauryl | | | | | | |
| Alcohol²⁾ | | | | | | |
| alkyl | | | 8.30% | 7.7% | | |
| polyglucoside | | | | | | |
| C8-C10³⁾ | | | | | | |
| Deceth-3¹⁾ | 1.50% | | | | | |
| Lauryl lactate | | | | 1% | | |
| Lauric acid | | 1.5% | 1.60% | | | |
| Glyceryl | | | | | 3.00% | 3.00% |
| Caprylate | | | | | | |
| Fragrance | 3.00% | 3.0% | 3.00% | 3.00% | 3.00% | 0% |

¹⁾Deceth-8; trademark and origin: KLK Oleo
²⁾Laureth-9;; trademark and origin
³⁾Plantacare 2000UP; trademark and origin: BASF

Different ringing gel compositions are prepared (compositions 1-6) according to the following protocol.

In a first step, the aqueous phase (water), the solvent (propylene glycol) if present and surfactants are mixed together at room temperature under agitation with magnetic stirrer at 300 rpm for 5 min.

In a second step, the linker is dissolved in the hydrophobic active ingredient (fragrance) at room temperature under agitation with magnetic stirrer at 300 rpm. The resulting mixture is mixed for 5 min.

Then, the aqueous phase and the oil phase are mixed together at room temperature for 5 min leading to the formation of a transparent or opalescent ringing gel.

### Example 10

### Powder Detergent Composition

A sufficient amount of granules 1-5 is weighed and mixed in a powder detergent composition to add the equivalent of 0.2% perfume.

TABLE 7
Powder detergent composition

| Ingredients | Part |
|---|---|
| Anionic (Linear Alkyl Benzene | 20% |
| Sulphonates) | |
| Nonionics (Alcohol Ethoxylates (5-9 | 6% |
| ethylene oxide) | |
| Builders (zeolites, sodium carbonate) | 25% |
| Silicates | 6% |
| Sodium Sulphate | 35% |
| Others (Enzymes, Polymers, Bleach) | 7.5% |
| Spray-dried granule powder 1-5 | 0.5% |

### Example 11

### Concentrated all Purpose Cleaner Composition

A sufficient amount of microcapsule slurry A-E is weighed and mixed in a concentrated all-purpose cleaner composition to add the equivalent of 0.2% perfume.

TABLE 8
concentrated all-purpose cleaner composition

| Ingredients | Amount (% wt) | Function |
|---|---|---|
| Ethoxylated Alcohol (C9- | 20 | Non-ionic surfactant |
| C11, 8EO) ⁽¹⁾ | | |
| Sodium Dodecyl Benzene | 16 | Anionic surfactant |
| Sulfonate ⁽²⁾ | | |
| Sodium Cumene Sulfonate | 8 | Hydrotrope |
| ⁽³⁾ | | |
| Methyl chloro | 0.8% | preservative |
| isothiazolinone Methyl | | |
| isothiazolinone 3.3:1 ⁽⁴⁾ | | |
| Water | 55.9 | solvent |

⁽¹⁾ Neodol 91-8 ®; trademark and origin: Shell Chemical
⁽²⁾ Biosoft D-400; trademark and origin: Stepan Company
⁽³⁾ Stepanate SCS ®; trademark and origin: Stepan Company
⁽⁴⁾ Kathon CG ®; trademark and origin: Dow Chemical Company

All ingredients are mixed together and then the mixture was diluted with water to 100%.

### Example 12

### Solid Scent Booster Composition

A sufficient amount of microcapsules in dried form is weighed and mixed with a solid scent booster composition to add the equivalent of 0.2% perfume.

TABLE 9
Salt-based solid scent booster compositions

| Ingredients | Part |
|---|---|
| Sodium chloride | 95 |
| Spray-dried granule powder 1-5 | 5 |

TABLE 10
Urea-based solid scent booster compositions

| Ingredients | Part |
|---|---|
| Urea (beads) | 86 |
| Spray-dried granule powder 1-5 | 8 |
| Bentonite | 3 |
| Perfume | 3 |

### Example 13

### Shampoo Composition

A sufficient amount of microcapsule slurry A-E is weighed and mixed in a shampoo composition to add the equivalent of 0.2% perfume.

TABLE 11
Shampoo composition

| | | Concentration |
|---|---|---|
| | Ingredients | [wt %] |
| A | Water deionized | 44.4 |
| | Polyquaternium-10 ¹⁾ | 0.3 |
| | Glycerin 85% ²⁾ | 1 |
| | DMDM Hydantoin ³⁾ | 0.2 |
| B | Sodium Laureth Sulfate ⁴⁾ | 28 |
| | Cocamidopropyl Betaine ⁵⁾ | 3.2 |
| | Disodium Cocoamphodiacetate ⁶⁾ | 4 |
| | Ethoxy (20) Stearyl Alcohol ⁶⁾ | 1 |
| C | Sodium Laureth Sulfate ⁴⁾ | 3 |
| | Glyceryl Laureate ⁷⁾ | 0.2 |
| D | Water deionized | 1 |
| | Sodium Methylparaben ⁸⁾ | 0.1 |
| E | Sodium Chloride 10% aqueous sol. | 15 |
| | Citric acid 10% aqueous sol. till pH 5.5-6 | q.s. |
| | Perfume | 0.5 |
| | TOTAL: | 100 |

¹⁾ Ucare Polymer JR-400, Noveon
²⁾ Schweizerhall
³⁾ Glydant, Lonza
⁴⁾ Texapon NSO IS, Cognis
⁵⁾ Tego Betain F 50, Evonik
6) Amphotensid GB 2009, Zschimmer & Schwarz
7) Monomuls 90 L-12, Gruenau
8) Nipagin Monosodium, NIPA

Polyquaternium-10 is dispersed in water. The remaining ingredients of phase A are mixed separately by addition of one after the other while mixing well after each adjunction. Then this pre-mix is added to the Polyquaternium-10 dispersion and was mixed for 5 min. Then Phase B and the premixed Phase C (heat to melt Monomuls 90L-12 in Texapon NSO IS) are added. The mixture is mixed well. Then, Phase D and Phase E are added while agitating. The pH was adjusted with citric acid solution till pH: 5.5-6.0.

### Example 14

### Shampoo Composition

A sufficient amount of microcapsule slurry A-E is weighed and mixed in a shampoo composition to add the equivalent of 0.2% perfume.

TABLE 12
Shampoo composition

| | | Concen- |
|---|---|---|
| | | tration |
| | Ingredients | [wt %] |
| A | Water deionized | 45.97 |
| | Tetrasodium EDTA ¹⁾ | 0.05 |
| | Guar Hydroxypropyltrimonium Chloride ²⁾ | 0.05 |
| | Polyquaternium-10 ³⁾ | 0.075 |
| B | NaOH 10% aqueous sol. | 0.3 |
| C | Ammonium Lauryl Sulfate ⁴⁾ | 34 |
| | Ammonium Laureth Sulfate ⁵⁾ | 9.25 |
| | Cocamidopropyl Betaine ⁶⁾ | 2 |
| | Dimethicone (&) C12-13 Pareth-4 (&) C12-13 | 2.5 |
| | Pareth-23 (&) Salicylic Acid ⁷⁾ | |
| D | Cetyl Alcohol ⁸⁾ | 1.2 |
| | Cocamide MEA ⁹⁾ | 1.5 |
| | Glycol Distearate ¹⁰⁾ | 2 |
| E | Methylchloroisothiazolinone & Methylisothiazolinone | 0.1 |
| | ¹¹⁾ | |
| | D-Panthenol 75% ¹²⁾ | 0.1 |
| | Water deionized | 0.3 |
| F | Sodium Chloride 25% aqueous sol. | 0.6 |
| | TOTAL: | 100 |

¹⁾ EDETA B Powder, BASF
²⁾ Jaguar C14 S, Rhodia
³⁾ Ucare Polymer JR-400, Noveon
⁴⁾ Sulfetal LA B-E, Zschimmer & Schwarz
⁵⁾ Zetesol LA, Zschimmer & Schwarz
⁶⁾ Tego Betain F 50, Evonik
⁷⁾ Xiameter MEM-1691, Dow Corning
⁸⁾ Lanette 16, BASF
⁹⁾ Comperlan 100, Cognis
¹⁰⁾ Cutina AGS, Cognis
¹¹⁾ Kathon CG, Rohm & Haas
¹²⁾ D-Panthenol, Roche

A premix comprising Guar Hydroxypropyltrimonium Chloride and Polyquaternium-10 are added to water and Tetrasodium EDTA while mixing. When the mixture is homogeneous, NaOH is added. Then, Phase C ingredients are added and the mixture was heat to 75° C. Phase D ingredients are added and mixed till homogeneous. The heating is stopped and temperature of the mixture is decreased to RT. At 45° C., ingredients of Phase E while mixing final viscosity is adjusted with 25% NaCl solution and pH of 5.5-6 is adjusted with 10% NaOH solution.

### Example 15

### Antiperspirant Spray Anhydrous Composition

A sufficient amount of microcapsule slurry microcapsule slurry A-E is weighed and mixed in an antiperspirant spray anhydrous composition to add the equivalent of 0.2% perfume.

TABLE 13
antiperspirant spray anhydrous composition

| Ingredient | Amount (wt %) |
|---|---|
| Cyclomethicone¹⁾ | 53.51 |
| Isopropyl miristate | 9.04 |
| Silica²⁾ | 1.03 |
| Quaternium-18-Hectorite³⁾ | 3.36 |
| Aluminium Chlorohydrate⁴⁾ | 33.06 |

¹⁾Dow Corning ® 345 Fluid; trademark and origin: Dow Corning
²⁾Aerosil ® 200 ; trademark and origin: Evonik
³⁾Bentone ® 38; trademark and origin: Elementis Specialities
⁴⁾Micro Dry Ultrafine; origin: Reheis

Using a high speed stirrer, Silica and Quaternium-18-Hectorite are added to the Isopropyl miristate and Cyclomethicone mixture. Once completely swollen, Aluminium Chlorohydrate is added portion wise under stirring until the mixture was homogeneous and without lumps. The aerosol cans are filled with 25% Suspension of the suspension and 75% of Propane/Butane (2,5 bar).

### Example 16

### Antiperspirant Spray Emulsion Composition

A sufficient amount of microcapsule slurry microcapsule slurry A-E is weighed and mixed in antiperspirant spray emulsion composition to add the equivalent of 0.2% perfume.

TABLE 14
antiperspirant spray emulsion composition

| Ingredient | Amount (wt %) |
|---|---|
| Polysorbate 65¹⁾ (Part A) | 0.95 |
| Polyglyceryl-2 dipolyhydroxystearate²⁾ | 1.05 |
| (Part A) | |
| Cetyl PEG/PPG-10/1 Dimethicone³⁾ | 2.75 |
| (Part A) | |
| Cyclomethicone⁴⁾ (Part A) | 16.4 |
| Isopropylisostearate⁵⁾ (Part A) | 4.5 |
| Phenoxyethanol⁶⁾ (Part A) | 0.5 |
| Ethylhexylglycerin⁷⁾ (Part A) | 0.2 |
| C12-15 Alkyl Benzoate⁸⁾ (Part A) | 5.65 |
| Silica Silylate⁹⁾ (Part A) | 0.1 |
| Sodium Methylparaben¹⁰⁾ (Part B) | 0.1 |
| Aluminium Chlorohydrate¹¹⁾ (Part B) | 20 |
| Water (Part B) | 44.47 |
| Fragrance (Part C) | 3.33 |

¹⁾Tween 65; trademark and origin: CRODA
²⁾Dehymuls PGPH; trademark and origin: BASF
³⁾Abil EM-90; trademark and origin: BASF
⁴⁾Dow Corning 345 fluid; trademark and origin: Dow Corning
⁵⁾Crodamol ipis; trademark and origin: CRODA
⁶⁾Phenoxyethanol; trademark and origin: LANXESS
⁷⁾Sensiva sc 50; trademark and origin: KRAFT
⁸⁾Tegosoft TN; trademark and origin: Evonik
⁹⁾Aerosil R 812; trademark and origin: Evonik
¹⁰ Nipagin mna; trademark and origin: CLARIANT
¹¹⁾Locron L; trademark and origin: CLARIANT

The ingredients of Part A and Part B are weighted separately. Ingredients of Part A are heated up to 60° C. and ingredients of Part B are heated to 55° C. Ingredients of Part B are poured small parts while continuous stirring into A. Mixture were stirred well until the room temperature was reached. Then, ingredients of part C are added. The emulsion is mixed and is introduced into the aerosol cans. The propellant is crimped and added.

Aerosol filling: 30% Emulsion: 70% Propane/Butane 2,5 bar

### Example 17

### Deodorant Spray Composition

A sufficient amount of microcapsule slurry microcapsule slurry A-E is weighed and mixed in antiperspirant deodorant spray composition to add the equivalent of 0.2% perfume.

TABLE 15
deodorant spray composition

| Ingredient | Amount (wt %) |
|---|---|
| Ethanol 95% | 90.65 |
| Triclosan¹⁾ | 0.26 |
| Isopropyl miristate | 9.09 |

¹⁾Irgasan ® DP 300; trademark and origin: BASF

All the ingredients according to the sequence of the Table above are mixed and dissolved. Then the aerosol cans are filled, crimp and the propellant is added (Aerosol filling: 40% active solution 60% Propane/Butane 2.5 bar).

### Example 18

### Antiperspirant Roll-on Emulsion Composition

A sufficient amount of microcapsule slurry microcapsule slurry A-E is weighed and mixed in antiperspirant roll-on emulsion composition to add the equivalent of 0.2% perfume.

TABLE 16
antiperspirant roll-on emulsion composition

| Ingredient | Amount (wt %) |
|---|---|
| Steareth-2¹⁾ (Part A) | 3.25 |
| Steareth-21²⁾ (Part A) | 0.75 |
| PPG-15 Stearyl Ether³⁾ (Part A) | 4 |
| WATER deionised (Part B) | 51 |
| Aluminum Chlorohydrate 50% | 40 |
| aqueous solution⁴⁾ (Part C) | |
| Fragrance (Part D) | 1 |

¹⁾BRIJ 72; origin: ICI
²⁾BRIJ 721; origin: ICI
³⁾ARLAMOL E; origin: UNIQEMA-CRODA
⁴⁾LOCRON L; origin: CLARIAN

Part A and B are heated separately to 75° C.; Part A is added to part B under stirring and the mixture is homogenized for 10 minutes. Then, the mixture is cooled down under stirring; and part C is slowly added when the mixture reached 45° C. and part D when the mixture reached at 35° C. while stirring. Then the mixture is cooled down to RT.

### Example 19

### Antiperspirant Roll-on Composition

A sufficient amount of microcapsule slurry microcapsule slurry A-E is weighed and mixed in antiperspirant roll-on composition to add the equivalent of 0.2% perfume.

TABLE 17
antiperspirant roll-on composition

| Ingredient | QUANTITY |
|---|---|
| Water (Part A) | 45 |
| Aluminum Chlorohydrate 50% aqueous | 20 |
| solution¹⁾ (Part B) | |
| Alcohol Denat. (Ethanol 96%) (Part B) | 30 |
| Ceteareth-12²⁾ (Part C) | 2 |
| Ceteareth-30³⁾ (Part C) | 2 |
| Fragrance (Part D) | 1 |

¹⁾LOCRON L; origin: CLARIANT
²⁾EUMULGIN B-1; origin: BASF
³⁾EUMULGIN B-3; origin: BASF

The ingredients of part B are mixed in the vessel then ingredient of part A is added. Then dissolved part C in part A and B. With perfume, 1 part of Cremophor RH40 for 1 part of perfume is added while mixing well

### Example 20

### Antiperspirant Roll-on Composition

A sufficient amount of microcapsule slurry microcapsule slurry A-E is weighed and mixed in antiperspirant roll-on emulsion composition to add the equivalent of 0.2% perfume.

TABLE 18
antiperspirant roll-on emulsion composition

| Ingredient | Amount (wt %) |
|---|---|
| Water (Part A) | 50.51 |
| Hydroxyethylcellulose¹⁾ (Part A) | 0.71 |
| Ethanol 95% (Part B) | 40.40 |
| 1,2-Propylene Glycol (Part B) | 5.05 |
| Triclosan²⁾ (Part B) | 0.30 |
| PEG-40 Hydrogenated castor oil³⁾ (Part C) | 3.03 |

¹⁾Natrosol ® 250 H; trademark and origin: Ashland
²⁾Irgasan ® DP 300; trademark and origin: BASF
³⁾Cremophor ® RH 40; trademark and origin: BASF

Part A is prepared by sprinkling little by little the Hydroxyethylcellulose in the water whilst rapidly stirring with the turbine. Stirring is continued until the Hydroxyethylcellulose is entirely swollen and giving a limpid gel. Then, Part B is poured little by little in Part A whilst continuing stirring until the whole is homogeneous. Part C is added.

### Example 21

### Deodorant Pump without Alcohol Formulation

A sufficient amount of microcapsule slurry microcapsule slurry A-E is weighed and mixed in the following composition to add the equivalent of 0.2% perfume.

TABLE 19
deodorant composition

| Ingredients | Amount (wt %) |
|---|---|
| C12-15 Alkyl | 5 |
| Lactate¹⁾ | |
| Dimethicone²⁾ | 91.6 |
| Cetyl Lactate³⁾ | 1 |
| Octyldodecanol⁴⁾ | 0.8 |
| Triclosan⁵⁾ | 0.1 |
| PERFUME | 1.5 |

¹⁾Ceraphyl 41; trademark and origin ASHLAND
²⁾DOW CORNING 200 FLUID 0.65cs; trademark and origin DOW CORNING CORPORATION
³⁾Ceraphyl 28; trademark and origin ASHLAND
⁴⁾Eutanol G; trademark and origin BASF
⁵⁾Irgasan@ DP 300; trademark and origin: BASF

All the ingredients are mixed according to the sequence of the table and the mixture is heated slightly to dissolve the Cetyl Lactate.

### Example 22

### Deodorant Pump with Alcohol Formulation

A sufficient amount of microcapsule slurry microcapsule slurry A-E is weighed and mixed in the following composition to add the equivalent of 0.2% perfume.

TABLE 20
deodorant composition

| | Amount |
|---|---|
| Ingredients | (wt %) |
| Ethyl Alcohol (Part A) | 60 |
| PEG-6 Caprylic/Capric Glycerides¹⁾ (Part A) | 2 |
| Water (Part A) | 35.6 |
| PEG-40 Hydrogenated Castor Oil²⁾ (Part B) | 0.4 |
| PERFUME (Part B) | 2 |

¹⁾Softigen 767; trademark and origin CRODA
²⁾Cremophor ® RH 40; trademark and origin: BASF

Ingredients from Part B are mixed together. Ingredients of Part A are dissolved according to the sequence of the Table and are poured into part B.

### Example 23

### Deodorant Stick without Alcohol Formulation

A sufficient amount of microcapsule slurry microcapsule slurry A-E is weighed and mixed in the following composition to add the equivalent of 0.2% perfume.

TABLE 21
deodorant composition

| Ingredient | Amount (wt %) |
|---|---|
| Stearic acid (Part A) | 5.05 |
| 1,2-propylene glycol (Part A) | 41.87 |
| Sodium hydroxide 20% aqueous solution | 4.24 |
| (Part A) | |
| Water (Part A) | 30.30 |
| Tetrasodium EDTA¹⁾ (Part A) | 0.10 |
| Ceteareth-25²⁾ (Part A) | 1.52 |
| PPG-3 Myristyl ether³⁾ (Part A) | 1.52 |
| 1,2-propylene glycol (Part B) | 15.14 |
| Triclosan⁴⁾ (Part B) | 0.25 |

¹⁾Edeta ® B Power; trademark and origin: BASF
²⁾Cremophor ® A25; trademark and origin: BASF
³⁾Tegosoft ® APM; trademark and origin: Evonik
⁴⁾Irgasan ® DP 300; trademark and origin: BASF

All the components of Part A are weighted and heated up to 70-75° C. Ceteareth-25 is added once the other Part A ingredients are mixed and heated. Once the Ceteareth-25 is dissolved, the Stearic Acid is added. Part B is prepared by dissolving the Triclosan in 1,2 Propylene Glycol. Water which has evaporated is added. Slowly under mixing, Part B is poured into part A. To stock, a plastic bag into the bucket is put in to be sealed after cooling. Moulds was filled at about 70° C.

### Example 24

### Anti-Perspirant Stick

A sufficient amount of microcapsule slurry microcapsule slurry A-E is weighed and mixed in the following composition to add the equivalent of 0.2% perfume.

TABLE 22
deodorant composition

| Ingredient | Amount (wt %) |
|---|---|
| Cyclomethicone¹⁾ (Part A) | 55.56 |
| Stearyl Alcohol²⁾ (Part A) | 21.21 |
| PPG-14 Butyl ether³⁾ (Part A) | 2.02 |
| Hydrogenated Castor Oil⁴⁾ (Part A) | 1.01 |
| Aluminium Zirconium tetrachlorohydrex- | 20.20 |
| Gly⁵⁾ (Part B) | |

¹⁾Dow Corning ® 345 Fluid; trademark and origin: Dow Corning
²⁾Lanette ® 18; trademark and origin: BASF
³⁾Tegosoft ® PBE; trademark and origin: Evonik
⁴⁾Cutina ® HR; trademark and origin: BASF
⁵⁾Summit AZP-908; trademark and origin: Reheis

All the components of Part A are weighted, heated up to 70-75° C. and mixed well. Ingredient of Part B is dispersed in Part A. The mixture is mixed and putted into a tick at 65° C.

### Example 25

### Day Cream

A sufficient amount of microcapsule slurry microcapsule slurry A-E is weighed and mixed in the following composition to add the equivalent of 0.2% perfume.

TABLE 23
day cream

| Ingredients | % |
|---|---|
| ARLATONE 985 | 5.000 |
| Ethoxylated Fatty Alcohol Ester | |
| CETYL ALCOHOL | 0.500 |
| TEFOSE 2561 | 4.000 |
| Ceteth-20 (and) Glyceryl Stearate | |
| (and) PEG-6 Stearate (and) Steareth-20 | |
| COSBIOL | 1.000 |
| Squalan | |
| MINERAL OIL 30-40 cp | 2.000 |
| Paraffin Oil | |
| PETROLEUM JELLY | 6.000 |
| Petrolatum | |
| WATER deionized | 75.850 |
| PROPYLENE GLYCOL | 5.000 |
| GLYDANT PLUS | 0.150 |
| DMDM Hydantoin (and) | |
| Iodopropynyl Butylcarbamate | |
| PNC 400 | 0.200 |
| Sodium Carbomer | |
| PERFUME | 0.300 |
| Total | 100.00 |

### Example 26

### Talc Formulation

A sufficient amount of granules 1-5 is weighed and mixed in introduced in a standard talc base: 100% talc, very slight characteristic odor, white powder, origin: LUZENAC to add the equivalent of 0.2% perfume.

### Example 27

### Shower-Gel Composition

A sufficient amount of microcapsule slurry microcapsule slurry A-E is weighed and mixed in the following composition to add the equivalent of 0.2% perfume.

TABLE 24
shower gel composition

| Ingredients | Amount (% wt) | Function |
|---|---|---|
| WATER deionised | 49.350 | Solvent |
| Tetrasodium EDTA ¹⁾ | 0.050 | Chelating agent |
| Acrylates Copolymer²⁾ | 6.000 | Thickener |
| Sodium C12-C15 Pareth Sulfate ³⁾ | 35.000 | Surfactant |
| Sodium Hydroxide 20% | 1.000 | pH adjuster |
| aqueous solution | | |
| Cocamidopropyl Betaine⁴⁾ | 8.000 | Surfactant |
| Methylchloroisothiazolinone | 0.100 | Preservative |
| and Methylisothiazolinone⁵⁾ | | |
| Citric Acid (40%) | 0.500 | pH adjuster |

¹⁾ EDETA B POWDER; trademark and origin: BASF
²⁾CARBOPOL AQUA SF-1 POLYMER; trademark and origin: NOVEON
³⁾ ZETESOL AO 328 U; trademark and origin: ZSCHIMMER & SCHWARZ
⁴⁾TEGO-BETAIN F 50; trademark and origin: GOLDSCHMIDT
⁵⁾KATHON CG; trademark and origin: ROHM & HASS

Ingredients are mixed, pH is adjusted to 6-6.3 (Viscosity: 4500 cPo+/−1500 cPo (Brookfield RV/Spindle #4/20 RPM)).

### Example 28

### Shower-Gel Composition

A sufficient amount of microcapsule slurry microcapsule slurry A-E is weighed and mixed in the following composition to add the equivalent of 0.2% perfume.

TABLE 25
shower gel composition

| Ingredients | Amount (% wt) | Function |
|---|---|---|
| WATER deionized | 52.40 | Solvent |
| Tetrasodium EDTA ¹⁾ | 0.10 | Chelating agent |
| Sodium Benzoate | 0.50 | Preservative |
| Propylene Glycol | 2.00 | Solvent |
| Sodium C12-C15 Pareth Sulfate ²⁾ | 35.00 | Surfactant |
| Cocamidopropyl Betaine³⁾ | 8.00 | Surfactant |
| Polyquaternium-7⁴⁾ | 0.20 | Conditioning agent |
| Citric Acid (40%) | 1.00 | pH adjuster |
| Sodium Chloride | 0.80 | Viscosity adjuster |

¹⁾ EDETA B POWDER; trademark and origin: BASF
²⁾ ZETESOL AO 328 U; trademark and origin: ZSCHIMMER & SCHWARZ
³⁾TEGO-BETAIN F 50; trademark and origin: GOLDSCHMIDT
⁴⁾MERQUAT 550; trademark and origin: LUBRIZOL

Ingredients are mixed, pH is adjusted to 4.5 (Viscosity: 3000 cPo+/−1500 cPo (Brookfield RV/Spindle #4/20 RPM)).

### Example 29

### Shower-Gel Composition

A sufficient amount of microcapsule slurry microcapsule slurry A-E is weighed and mixed in the following composition to add the equivalent of 0.2% perfume.

TABLE 26
shower gel composition

| Ingredients | Amount (% wt) | Function |
|---|---|---|
| WATER deionized | 50.950 | Solvent |
| Tetrasodium EDTA ¹⁾ | 0.050 | Chelating agent |
| Sodium Benzoate | 0.50 | Preservative |
| Glycerin 86% | 3.50 | Solvent |
| Sodium Laureth Sulfate ²⁾ | 27.0 | Surfactant |
| Polyquaternium-7³⁾ | 1.0 | Conditioning Agent |
| Coco-Betaine⁴⁾ | 6.0 | Surfactant |
| PEG-120 Methyl Glucose trioleate⁵⁾ | 1.0 | Thickener |
| Citric Acid (40%) | 1.0 | pH adjuster |
| Glycol Distearate & Laureth-4 | 3.0 | Pearlizing agent |
| & Cocamidopropyl Betaine⁶⁾ | | |
| Sodium Chloride 20% | 5.0 | Viscosity adjuster |
| PEG-40 Hydrogenated Castor Oil⁷⁾ | 1.0 | Viscosity adjuster |

¹⁾ EDETA B POWDER; trademark and origin: BASF
²⁾ Texapon NSO IS; trademark and origin: COGNIS
³⁾MERQUAT 550; trademark and origin: LUBRIZOL
⁴⁾DEHYTON AB-30; trademark and origin: COGNIS
⁵⁾GLUCAMATE LT; trademark and origin: LUBRIZOL
⁶⁾EUPERLAN PK 3000 AM; trademark and origin: COGNIS
⁷⁾CREMOPHOR RH 40; trademark and origin: BASF

Ingredients are mixed, pH is adjusted to 4.5 (Viscosity: 4000 cPo+/−1500 cPo (Brookfield RV/Spindle #4/20 RPM))

### Example 30

### Hair Coloration Composition

A sufficient amount of microcapsule slurry microcapsule slurry A-E is weighed and mixed with alkaline base A to add the equivalent of 0.2% perfume.

2 g of alkaline base A is then mixed with 2 g of the oxidative base B

TABLE 27
Composition of the alkaline base A

| Ingredients | % |
|---|---|
| Phase A | |
| Water | 39.13 |
| Carbomer ¹⁾ | 0.9 |
| p-phenylenediamine ²⁾ | 1 |
| m-aminophenol ³⁾ | 1 |
| m-phenylenediamine sulfate ⁴⁾ | 0.07 |
| resorcinol ⁵⁾ | 0.5 |
| Phase B | |
| Propylene glycol | 6 |
| Laureth-2 ⁶⁾ | 6 |
| Laureth-12 ⁷⁾ | 6 |
| Dimethicone ⁸⁾ | 0.7 |
| Phase C | |
| Cetearyl alcohol ⁹⁾ | 18 |
| Oleth-30 ¹⁰⁾ | 3 |
| Lauric acid | 3 |
| Glycol distearate ¹¹⁾ | 3 |
| Phase D | |
| Sodium metabisulfite ¹²⁾ | 0.4 |
| Silica dimethyl silyate | 0.3 |
| Pentasodium pentetate ¹³⁾ | 0.2 |
| Polyquaternium-22 ¹⁴⁾ | 1 |
| Ammonium Hydroxide ¹⁵⁾ | 9.3 |
| Perfume | 0.5 |

¹⁾ Carbopol Ultrez 10 Polymer
²⁾ Covastyle PAP
³⁾ Covastyle MAP
⁴⁾ Covastyle MPDS
⁵⁾ Resorcine
⁶⁾ Lipocol L 12
⁷⁾ Arlypon F
⁸⁾ Dow Corning 200 Fluid 350
⁹⁾ Lanette O
¹⁰⁾ Eumulgin O 30
¹¹⁾ Cutina AGS
¹²⁾ Covastyle MBS
¹³⁾ Dissolvine D-40
¹⁴⁾ Merquat 280
¹⁵⁾ Ammonium hydroxide 30% aqueous solution

### Procedure:

All ingredients of Phase A were mixed and heated until 75° C.

All ingredients of Phase B were combined and melt at 70-75° C.

Phase B was added to Phase A (both at 70-75° C.) with good agitation.

Phase C was added while mixing continued until cooled down to room temperature

At room temperature Phase D ingredients were added while mixing Remaining ingredients of Phase C were added under stirring.

TABLE 28
Composition of the oxidative base B

| Ingredients | % |
|---|---|
| Phase A | |
| Water | 75 |
| Phase B | |
| Cetearyl alcohol and dicethyl phospahate and | 3.5 |
| Cteteth-20 phosphate ¹⁾ | |
| Mineral oil ²⁾ | 3.5 |
| Cetyl acetate and acetylated lanolin alcohol ³⁾ | 0.35 |
| Steareth-20 ⁴⁾ | 0.35 |
| Phase C | |
| Hydrogen peroxide ⁵⁾ | 17 |
| Perfume | 0.3 |

¹⁾ Crodafos CS 20 Acid
²⁾ Paraffin Oil 30-40 cPs
³⁾ Acetulan
⁴⁾ Brij 78P
⁵⁾ Hydrogen Peroxide 35% aqueous solution

### Procedure:

All ingredients of Phase A were mixed and heated until 75° C.

All ingredients of Phase B were combined and melt at 70-75° C.

Phase B was added to Phase A (both at 70-75° C.) with good agitation and mixing continued until cooled down to room temperature

At room temperature Phase C ingredients were added while mixing

### Example 31

### Hand Dishwash

A sufficient amount of microcapsule slurry microcapsule slurry A-E is weighed and mixed in the following composition to add the equivalent of 0.2% perfume.

TABLE 29
Hand dishwash composition

| Ingredients | Amount (% wt) | Function |
|---|---|---|
| Linear alkylbenzene sulfonic | 20 | Anionic surfactant |
| acid ⁽¹⁾ | | |
| Diethanolamide ⁽²⁾ | 3.5 | Foam booster |
| Sodium Hydroxide (50%) ⁽³⁾ | 3.4 | pH Adjuster/neutralizer |
| Secondary alcohol | 2.5 | Non-ionic surfactant |
| ethoxolate ⁽⁴⁾ | | |
| Sodium xylene sulfonate | 6.3 | Hydrotrope |
| Water | 64.3 | Solvent |

¹⁾ Biosoft S-118 ®; trademark and origin: Stepan Company
²⁾ Ninol 40-CO ®; trademark and origin: Stepan Company
³⁾ Stepanate SXS ®; trademark and origin: Stepan Company
⁴⁾ Tergitol 15-S-9 ®; trademark and origin: Dow Chemical Company

Water with sodium hydroxide and diethanolamide are mixed. LAS is added. After the LAS is neutralized, the remaining ingredients are added. The pH was Checked (=7-8) and adjusted if necessary.

### Example 32

### Unit Dose Formulation

A sufficient amount of exemplified microcapsules is weighed and mixed in a unit dose formulation to add the equivalent of 0.2% perfume.

The unit dose formulation can be contained in a PVOH (polyvinyl alcohol) film.

TABLE 30
Unit dose composition

| Ingredients | Concentration [wt %] |
|---|---|
| C12-C14 alkyl poly ethoxylate | 15 |
| C12-C14 alkyl poly ethoxylate | 9.5 |
| sulfate Mono Ethanol Amine salt | |
| Linear Alkylbenzene sulfonic acid | 17 |
| Citric Acid | 0.5 |
| C12-C18 Fatty Acid | 17 |
| Enzymes | 1.2 |
| Fluorescent brightener | 0.3 |
| 1,2 propanediol | 12 |
| Glycerol | 9 |
| Sodium Hydroxide | 1 |
| Mono Ethanol Amine | 6 |
| PDMS | 2.5 |
| Potassium sulphite | 0.2 |
| Water | 8.8 |
| Total | 100 |

### Example 33

### Toothpaste Formulation

A sufficient amount of microcapsule slurry R (corresponding to microcapsules microcapsule slurry A-E except that a flavor is encapsulated instead of a perfume) is weighed and mixed in the following composition to add the equivalent of 0.2% flavor.

TABLE 31
Toothpaste formulation

| Ingredients | Amount (% wt) |
|---|---|
| Polyethylene glycol 400 | 2.0% |
| Xanthan Gum | 0.60% |
| Sorbitol 70% Solution | 50.0% |
| Sodium Fluoride | 0.220% |
| Sodium Benzoate | 0.20% |
| Water | 15.230% |
| Hydrated Silica¹⁾ | 22.0% |
| Hydrated Silica²⁾ | 7.0% |
| Titanium Dioxide CI77891 | 0.500% |
| Sodium Lauryl Sulfate | 1.250% |
| Flavor | 1.20% |
| TOTAL | 100% |

¹⁾Tixosil 73; trademark and origin:
²⁾Tixosil 43; trademark and origin:

### Example 34

### Dicalcium Phosphate Based Toothpaste Formulation

A sufficient amount of microcapsule slurry R (corresponding to microcapsules microcapsule slurry A-E except that a flavor is encapsulated instead of a perfume) is weighed and mixed in the following composition to add the equivalent of 0.2% flavor.

TABLE 32
Toothpaste formulation

| Ingredients | Amount (% wt) |
|---|---|
| Sodium carboxymethyl cellulose | 1.20% |
| Flavor | 1.20% |
| DI/Purified Water | Q.S to Final Wt. |
| Sodium Lauryl Sulfate | 1.30% |
| Glycerine | 20.0% |
| Sodium Saccharin | 0.20% |
| Dicalcium phosphate dihydrate | 36.0% |
| Methylparaben | 0.200% |
| Silica¹⁾ | 3.0% |
| TOTAL | 100% |

¹⁾Aerosil ®200; trademark and origin:

### Example 35

### Mouthwash Alcohol Free Formulation

A sufficient amount of microcapsule slurry R (corresponding to microcapsules microcapsule slurry A-E except that a flavor is encapsulated instead of a perfume) is weighed and mixed in the following composition to add the equivalent of 0.2% flavor.

TABLE 33
Mouthwash formulation

| Ingredients | Amount (% wt) |
|---|---|
| Propylene Glycol | 10.0% |
| Flavor | 0.240% |
| DI/Purified Water | Q.S to Final Wt. |
| Poloxamer 407 NF | 0.240% |
| Sodium Lauryl Sulfate | 0.040% |
| Sorbitol 70% Solution | 10.0% |
| Sodium Saccharin | 0.030% |
| Glycerine | 3.0% |
| Sodium Benzoate | 0.10% |
| Sucralose | 0.020% |
| Benzoic Acid | 0.050% |
| TOTAL | 100 % |

### Example 36

### Mouthwash Formulation

A sufficient amount of microcapsule slurry R (corresponding to microcapsules microcapsule slurry A-E except that a flavor is encapsulated instead of a perfume) is weighed and mixed in the following composition to add the equivalent of 0.2% flavor.

TABLE 34
Mouthwash formulation

| Ingredients | Amount (% wt) |
|---|---|
| Ethyl Alcohol 190 Proof | 15.00% |
| Flavor | 0.24% |
| DI/Purified Water | Q.S to Final Wt. |
| Poloxamer 407 NF | 0.24% |
| Sodium Lauryl Sulfate | 0.04% |
| Sorbitol 70% Solution | 10.00% |
| Sodium Saccharin | 0.03% |
| Glycerine | 3.00% |
| Sodium Benzoate | 0.10% |
| Sucralose | 0.02% |
| Benzoic Acid | 0.05% |
| TOTAL | 100% |

1. A polyamide core-shell microcapsule comprising:
(i) a core comprising a hydrophobic material, and
(ii) a polyamide shell comprising a reaction product between at least one acyl chloride and at least one co-oligopeptide.

2. The polyamide core-shell microcapsule according to claim 1, wherein the acyl chloride has the following formula (I) 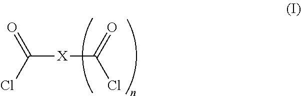
wherein n is an integer varying between 1 and 8, and
wherein X is an (n+1)-valent C₂ to C₄₅ hydrocarbon group optionally comprising at least one group selected from the group consisting of (i) to (xi), 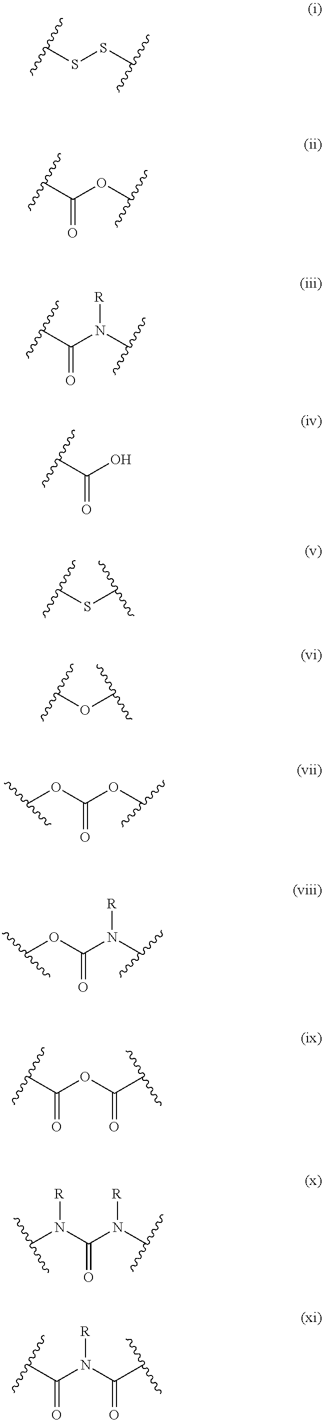
wherein R is a hydrogen atom or an alkyl group.

3. The polyamide core-shell microcapsule according to claim 2, wherein the acyl chloride is a diacyl chloride.

4. The polyamide core-shell microcapsule according to claim 1, wherein the co-oligopeptide has an average molecular weight less than 10,000 g/mol.

5. The polyamide core-shell microcapsule according to claim 1, wherein the co-oligopeptide comprises at least two different amino acids A and B, wherein the amino acid A is selected from the group consisting of lysine, arginine, ornithine, histidine, tryptophan, and mixtures thereof, and wherein the amino acid B is selected from the group consisting of lysine, arginine, ornithine, histidine, tryptophan, aspartic acid, glutamic acid, serine, threonine, asparagine, glutamine, glycine, proline, selenocysteine, alanine, valine, isoleucine, leucine, methionine, phenylalanine, tyrosine, citrulline, cysteine, and mixtures thereof.

6. The polyamide core-shell microcapsule according to claim 5, wherein the amino acid A is lysine.

7. The polyamide core-shell microcapsule according to claim 1, wherein the mole ratio of amino groups on average from the co-oligopeptide to acyl chloride groups from the acyl chloride is 0.05:1 to 65:1.

8. The polyamide core-shell microcapsule according to claim 1, wherein the hydrophobic material comprises a perfume.

9. A process for preparing a polyamide core-shell microcapsule slurry comprising:
a) dispersing an oil phase comprising a hydrophobic material and at least one acyl chloride into a dispersing phase to form a two-phases dispersion;
b) Performing a curing step to form microcapsules in the form of a slurry;
wherein at least one stabilizer is added in the oil phase and/or in the dispersing phase, and
wherein at least one co-oligopeptide is added in the dispersing phase and/or in the oil phase and/or in the two-phases dispersion.

10. The process according to claim 9, wherein a salt is added in the dispersing phase and/or in the oil phase and/or in the two-phases dispersion.

11. The process according to claim 9, wherein the stabilizer is selected from the group consisting of inorganic particles, polymeric emulsifiers, polysaccharides, proteins, glycoproteins, and mixtures thereof.

12. The process according to claim 9, wherein the acyl chloride is used in a concentration between 0.1% to 50% by weight based on the oil phase.

13. The process according to claim 9, wherein an amino-acid is added in the dispersing phase and/or the two-phases dispersion.

14. A consumer product comprising the microcapsules as defined in claim 1.

15. The microcapsule according to claim 2, wherein n is an integer between 1 and 6.

16. The microcapsule according to claim 1, wherein the acyl chloride is selected from the group consisting of terephthaloyl chloride, phthaloyl chloride, isophthaloyl chloride, adipoyl chloride, sebacoyl chloride, succinyl chloride, glutaryl chloride, pimeloyl chloride, dodecanedioyl dichloride, suberoyl chloride, azelaoyl chloride, malonyl chloride, fumaryl chloride, oxyalyl chloride, 4,4′-oxybis(benzoyl chloride), 2,2′-oxydiacetyl chloride, 4,4′-biphenyldicarbonyl chloride, azobenzene-4,4′-dicarbonyl dichloride, 2,5-furandicarbonyl dichloride, and mixtures thereof.

17. The microcapsule according to claim 1, wherein the mole ratio of amino group on average from co-oligopeptide to acyl chloride group from the acyl chloride is 0.1:1 to 10:1.

18. The process according to claim 9, wherein the acyl chloride concentration is between 0.5% to 15% by weight based on the oil phase.

19. The process according to claim 9, wherein an amino-acid selected from the group consisting of lysine, arginine, histidine, tryptophan, ornithine, glutamine, asparagine, citrulline, and mixtures thereof is added in the dispersing phase and/or the two-phases dispersion.

20. The consumer product of claim 14, wherein the consumer product is a home-care product, personal care product, or a fabric care product.
